(19) United States
(12) Patent Application Publication
(10) Pub. No.: US 2005/0054663 A1
(43) Pub. Date: Mar. 10, 2005

(54) GSK-3 inhibitors

(76) Inventors: Christina N. Bennett, Ann Arbor, MI (US); Kurt D. Hankenson, Ann Arbor, MI (US); Stephen D. Harrison, Albany, CA (US); Kenneth A. Longo, Ann Arbor, MI (US); Ormond A. MacDougald, Ypsilanti, MI (US); Allan S. Wagman, Belmont, CA (US)

Correspondence Address: Chiron Corporation;Intellectual Property - R440, P.O. Box 8097, Emeryville, CA, 94662-8097, US

(21) Appl. No.: 10/917,707

(22) Filed: Aug. 13, 2004

Related U.S. Application Data
(60) Provisional application No. 60/494,859, filed on Aug. 13, 2003.

(51) Int. Cl. A61K 31/513; A61K 31/506

(52) U.S. Cl. 514/269; 514/275

(57) ABSTRACT

This invention relates to methods of treating or preventing bone loss by administering to a human or animal subject pyrimidine and pyridine derivatives that inhibit the activity of glycogen synthase kinase 3 (GSK3), to pharmaceutical compositions containing the compounds, and to the use of the compounds and compositions alone or in combination with other pharmaceutically active agents.

### CROSS-REFERENCE TO RELATED APPLICATIONS

This application claims the benefit of U.S. patent application Ser. No. 60/494,859, filed Aug. 13, 2003. The disclosure of the above provisional application is herein incorporated by reference in its entirety and for all purposes as if fully set forth herein.

### BACKGROUND OF THE INVENTION

1. Field of the Invention

This invention relates to methods of treating or preventing bone loss by administering to a human or animal subject pyrimidine and pyridine derivatives that inhibit the activity of glycogen synthase kinase 3 (GSK3). The invention further relates to pharmaceutical compositions containing the compounds and to the use of the compounds and compositions, either alone or in combination with other pharmaceutically active agents, in promoting bone formation.

2. State of the Art

### References

The following literature publications are cited in this section. All of the identified publications are herein incorporated by reference in their entirety to the same extent as if each individual publication was specifically and individually incorporated by reference in its entirety.
A. Asakura, M. Komaki, M. Rudnicki, Differentiation 68, 245-53 (2001).
A. I. Caplan, S. P. Bruder, Trends. Mol. Med. 7, 259-64 (2001).
M. E. Nuttall, J. M. Gimble, Bone 27, 177-84 (2000).
J. L. Kirkland, T. Tchkonia, T. Pirtskhalava, J. Han, I. Karagiannides, Exp. Gerontol. 37, 757-67 (2002).
S. E. Ross et. al., Science 953 (2000).
C. N. Bennett et. al., J. Biol. Chem. 277, 30998-1004 (2002).
R. T. Moon, B. Bowerman, M. Boutros, N. Perrimon, Science 296, 1644-6 (2002).
X. He, Dev. Cell 4, 791-7 (2003).
E. Smith, G. A. Coetzee, B. Frenkel, J. Biol. Chem., 277:20, pp. 18191-18197 (2002).
B. B. Kahn, J. S. Flier, J. Clin. Invest. 106, 473-481 (2000).
J. M. Taylor-Jones et. al., Mech. Ageing Dev. 123, 649-61 (2002).
G. K. Pavlath et. al., Dev. Dyn. 212, 495-508 (1998).
B. B. Lowell, B. M. Spiegelman, Nature 404, 652-60 (2000).
S. Enerback et. al., Nature 387, 90-4 (1997).
S. A. Thomas, R. D. Palmiter, Nature 387, 94-7 (1997).
J. Moitra et. al., Genes Dev. 12, 3168-3181 (1998).
I. Shimomura et. al., Genes & Devel. 12, 3182-3194 (1998).
E. D. Rosen, C. J. Walkey, P. Puigserver, B. M. Spiegelman, Genes Dev. 14, 1293-307 (2000).
G. Bain, T. Muller, X. Wang, J. Papkoff, Biochem. Biophys. Res. Commun. 301, 84-91 (2003).
Y. Gong et. al., Cell 107, 513-23 (2001).
L. M. Boyden et. al., N. Engl. J. Med. 346, 1513-21 (2002).
S. Takeda et. al., Cell 111, 305-17 (2002).
K. D. Hankenson et. al., J. Bone Miner. Res. 15, 851-62 (2000).
O. A. MacDougald, C.-S. Hwang, H. Fan, M. D. Lane, Proc. Natl. Acad. Sci. U.S.A. 92, 9034-9037 (1995).
K. D. Hankenson, P. Bornstein, J. Bone Miner. Res. 17, 415-25 (2002).
W. J. Boyle, W. S. Simonet, D.L. Lacey, Nature, 423, p.337-342 (2003).

Bone renewal, or remodeling, is an ongoing process in bone tissues involving both bone formation and bone resorption events that are respectively carried out by hematopoietically derived osteoblasts and osteoclasts. Disruption of this balance favoring bone resorption and osteoclastic activity is related to a number of pathologies including osteopenia, osteoporosis, steroid induced osteroporosis, periodontal disease, rheumatoid arthritis, and Paget's disease. Common drugs used to treat these conditions act as anti-resorption agents and include the peptide calcitonin and the bisphosphates alendronate, clodronate, etidronate, pamidronate, and tiludronate, and risedronate. However, effective agents for promoting osteogenesis, or bone formation, remain lacking. Potential drugs that directly stimulate bone formation are currently still in clinical trials. Teriparatide, a recombinant parathyroid hormone, is the only drug having a pro-bone forming mechanism of action that has been approved for the treatment of osteoporosis. Osteogenesis promoting agents would be particularly useful in initiating bone formation in conditions involving acute bone loss resulting from trauma or cancer.

Osteogenesis is dependent on mesenchymal progenitors. These cells can differentiate not only into osteoblasts but also into adipocytes, myoctes, and other cell types (Asakura et. al. 2001, and Caplan e. al. 2001). Wnts are a family of secreted signaling proteins that regulate many cellular events, including developmental processes. A reciprocal relationship exists between adipogenesis and differentiation to other lineages in vitro and in vivo, such that loss of bone or muscle is associated with increased number of adipocytes within those tissues (Nuttall et. al. 2000 and Kirkland, et. al. 2002). One potential regulator governing cell fate of multipotent mesenchymal progenitors is Wnt10b, which inhibits adipogenesis in vitro (Ross et. al., 2000 and Bennett et. al. 2002). In the canonical signaling pathway, secreted Wnts act through frizzled receptors and LRP coreceptors to inhibit glycogen synthase kinase 3, stabilize β-catenin, and influence activity of T-cell factor TCF/lymphoid-enhancing factor LEF transcription factors (Moon et. al. 2002 and He 2003). Activation of canonical Wnt signaling inhibits adipocyte conversion, and inhibition of Wnt signaling in preadipocytes causes spontaneous adipogenesis. The best candidate for the endogenous inhibitory Wnt is Wnt10b, which blocks adipocyte conversion and is expressed in precursor cells but not adipocytes. The GSK3 inhibitor CHIR99021, 6-[(2-{[4-(2,4-dichlorophenyl)-5-(4-methylimidazol-2-yl)pyrimidin-2-yl]amino}ethyl)amino]pyridine-3-carbonitrile disclosed in WO 99/65897, has been found to mimic Wnt signaling in vitro in 3T3-L1 preadipocyes by activating Wnt and consequently blocking adipocyte conversion (Bennett et. al. 2002).

A study of the effects of glucocorticoid steroids in promoting steroid induced osteoporosis indicated that the kinase GSK3β may play an key role in this disease by disrupting the osteoblast cell cycle through activation of GSK3β (Smith et. al. 2002). GSK3, also known as glycogen synthase kinase-3, is a serine/threonine kinase for which two isoforms, α and β, have been identified. The mechanism and specific pathway by which glucocorticoids exert their influence on GSK3β is unclear, as GSK3β itself participates in Wnt and growth factor pathways affecting a broad range of cellular function ranging from protein synthesis, cell proliferation, cell differentiation, and apoptosis to immune potentiation.

### BRIEF DESCRIPTION OF THE DRAWINGS

The foregoing aspects and many of the attendant advantages of this invention will become more readily appreciated by reference to the following summary and detailed description, when taken in conjunction with the accompanying figures.

FIG. 1A. Wnt10b increases trabecular bone and osteogenesis. Micro-computed tomography of femurs from wild type and FABP4-Wnt10b mice (upper panel) was performed as described (Hankenson et. al. 2000). Three-dimensional reconstruction of metaphyseal trabeculae from highlighted boxed region (lower panel).

FIG. 1B. Multipotential ST2 cells were induced to undergo osteogenesis as described (Hankenson and Bornstein 2002). On days 0 and 2, cells were treated with DMSO (control) or 3 μM CHIR99021 6-[(2-{[4-(2,4-dichlorophenyl)-5-(4-methylimidazol-2-yl)pyrimidin-2-yl]amino}ethyl)amino]pyridine-3-carbonitrile (Chiron Corporation, Emeryville, Calif.). On day 10, cells were stained with Alizarin Red-S for mineralization.

### SUMMARY OF THE INVENTION

The present invention provides compositions and methods for treating or preventing bone loss in a human or animal subject. In one aspect, the present invention provides compounds having following formula (I): 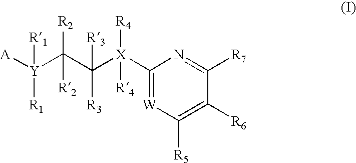
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R′₁, R′₂, R′₃ and R′₄ are independently selected from the group consisting of hydrogen, and optionally substituted loweralkyl;
R₅ and R₇ are independently selected from the group consisting of hydrogen, halo, and optionally substituted loweralkyl, cycloalkyl, alkoxy, amino, aminoalkoxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido; or
pharmaceutically acceptable salts thereof, stereoisomers thereof, tautomers thereof, hydrates thereof, or solvates thereof.

In some embodiments of the invention, compounds of formulas (IV) and (V) are provided: 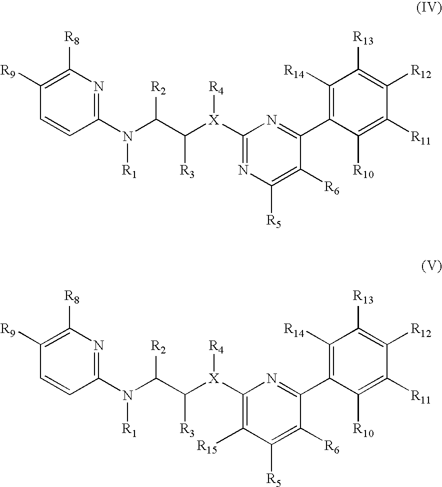
wherein X, R₁-R₆, and R₈-R₁₄ have the meanings described above, and R₅ is selected from the group consisting of hydrogen, nitro, cyano, amino, alkyl, halo, haloloweralkyl, alkyloxycarbonyl, aminocarbonyl, alkylsulfonyl and arylsulfonyl, or the pharmaceutically acceptable salts thereof, stereoisomers thereof, tautomers thereof, hydrates thereof, or solvates thereof.

Another aspect of this invention provides a method of treating or preventing a bone loss in a human or animal subject, comprising administering to the human or animal subject compounds disclosed herein, including compound (VI), or the pharmaceutically acceptable salts thereof, stereoisomers thereof, tautomers thereof, hydrates thereof, or solvates thereof, wherein compound (VI) is 6-[(2-{[4-(2,4-dichlorophenyl)-5-(4-methylimidazol-2-yl)pyrimidin-2-yl]amino}ethyl)amino]pyridine-3-carbonitrile and has the formula:

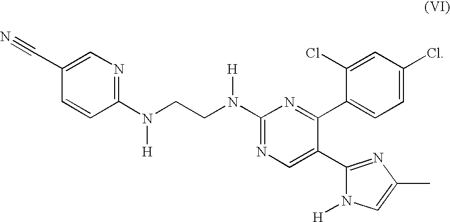

The bone loss treated or prevented by the administration of compounds of this invention include but are not limited to bone loss related to osteopenia, osteoporosis, drug therapy, postmenopausal bone loss, age, disuse, diet, rheumatism, rheumatoid arthritis, Paget's disease, periodontal disease, cancer, cancer treatment, or bone fracture. Bone loss occurring through steroid administration as part of a drug therapy regimen or through the use of cytotoxic agents during cancer treatment is also treated or prevented by the administration of compounds of the invention. Cancers and cancer treatments related to bone loss contemplated by the present invention include multiple myeloma, breast, prostate, or lung cancer.

Yet another aspect of this invention provides a method of increasing or promoting bone formation or bone growth by administering to the human or animal subject compounds of the invention having formula (I), (IV), (V), or (VI), or the pharmaceutically acceptable salts thereof, stereoisomers thereof, tautomers thereof, hydrates thereof, or solvates thereof.

This invention further provides a method of healing bone fractures by administration of a compound or the pharmaceutically acceptable salts thereof, stereoisomers thereof, tautomers thereof, hydrates thereof, or solvates thereof having formula (I), (IV), (V), or (VI) to a human or an animal subject. Any bone fracture, including fractures of the hip or spine, can be treated by administration of the compounds disclosed herein.

The present invention also provides a method for treating or preventing bone loss in a human or animal subject, comprising administering to the human or animal subject a compound or the pharmaceutically acceptable salts thereof, stereoisomers thereof, tautomers thereof, hydrates thereof, or solvates thereof having formula (I), (IV), (V), or (VI) in combination with at least one additional agent for the treatment or prevention of a bone loss.

The invention further provides a composition containing a compound, the pharmaceutically acceptable salts thereof, stereoisomers thereof, tautomers thereof, hydrates thereof, or solvates thereof having formula (I), (IV), (V), or (VI), and at least one additional agent for the treatment or prevention of bone loss.

The additional agents provided by the invention for use in the methods and compositions include estrogen, calcium, anti-resorption agents, raloxifene, calcitonin, alendronate, clodronate, etidronate, pamidronate, ibandronate, zoledronic acid, risedronate, and tiludronate. Also included are osteogenic promoting agents such as parathyroid hormone or recombinant or synthetic parathyroid hormone.

The invention also provides for use of a compound having formula (I), (IV), (V), or (VI) or the pharmaceutically acceptable salts thereof, stereoisomers thereof, tautomers thereof, hydrates thereof, or solvates thereof in the manufacture of a medicament for the prevention or treatment of bone loss.

The methods, compounds and compositions of the invention may be employed alone, or in combination with other pharmacologically active agents in the prevention or treatment of disorders mediated by GSK3 activity, such as in the treatment of diabetes, Alzheimer's disease and other neurodegenerative disorders, obesity, atherosclerotic cardiovascular disease, essential hypertension, polycystic ovary syndrome, syndrome X, ischemia, especially cerebral ischemia, traumatic brain injury, bipolar disorder, immunodeficiency or cancer.

### DETAILED DESCRIPTION

In accordance with the present invention, compounds, compositions, and methods are provided for the inhibition of glycogen synthase kinase 3 (GSK3) activity in the treatment or prevention of bone loss in a human or animal subject. In one aspect, the present invention provides compounds having formula (I): 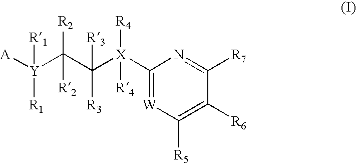
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R′₁, R′₂, R′₃ and R′₄ are independently selected from the group consisting of hydrogen, and optionally substituted loweralkyl;
R₅ and R₇ are independently selected from the group consisting of hydrogen, halo, and optionally substituted loweralkyl, cycloalkyl, alkoxy, amino, aminoalkoxy, alkylamino, aralkylamino, heteroaralkylamino, arylamino, heteroarylamino cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteraralkylcarbonyl, alkylcarbonyloxy, arylcarbonyloxy, aralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroaralkylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido; or
pharmaceutically acceptable salts thereof, stereoisomers thereof, tautomers thereof, hydrates thereof, or solvates thereof.

In one presently preferred embodiment of the invention, at least one of X and Y is nitrogen. Representative compounds of this group include those compounds in which one of X and Y is nitrogen and the other of X and Y is oxygen or optionally substituted carbon. Preferably, both X and Y are nitrogen.

The constituent A can be an aromatic ring having from 3 to 10 carbon ring atoms and optionally 1 or more ring heteroatoms. Thus, in one embodiment, A can be optionally substituted carbocyclic aryl. Alternatively, A is optionally substituted heteroaryl, such as, for example, substituted or unsubstituted pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, which may substituted with at least one and not more than 3 substitution groups. Representative substitution groups can be independently selected from the group consisting of, for example, nitro, amino, cyano, halo, thioamido, amidino, oxamidino, alkoxyamidino, imidino, guanidino, sulfonamido, carboxyl, formyl, loweralkyl, haloloweralkyl, loweralkoxy, haloloweralkoxy, loweralkoxyalkyl, loweralkylaminoloweralkoxy, loweralkylcarbonyl, loweraralkylcarbonyl, lowerheteroaralkylcarbonyl, alkylthio, aminoalkyl and cyanoalkyl.

In some embodiments of the invention, A has the formula: 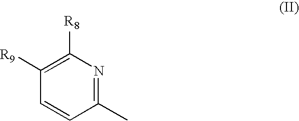
wherein R₈ and R₉ are independently selected from the group consisting of hydrogen, nitro, amino, cyano, halo, thioamido, amidino, oxamidino, alkoxyamidino, imidino, guanidinyl, sulfonamido, carboxyl, formyl, loweralkyl, haloloweralkyl, loweralkoxy, haloloweralkoxy, loweralkoxyalkyl, loweralkylaminoloweralkoxy, loweralkylcarbonyl, loweraralkylcarbonyl, lowerheteroaralkylcarbonyl, alkylthio, aryl and, aralkyl. Most preferably, A is selected from the group consisting of nitropyridyl, aminonitropyridyl, cyanopyridyl, cyanothiazolyl, aminocyanopyridyl, trifluoromethylpyridyl, methoxypyridyl, methoxynitropyridyl, methoxycyanopyridyl and nitrothiazolyl.

In other embodiments of the invention at least one of R₁, R₂, R₃ and R₄ may be hydrogen, or unsubstituted or substituted loweralkyl selected from the group consisting of haloloweralkyl, heterocycloaminoalkyl, and loweralkylaminoloweralkyl; or loweralkylaminoloweralkyl. Presently preferred embodiments of the invention include compounds wherein R₁, R₂, and R₃ are hydrogen and R₄ is selected from the group consisting of hydrogen, methyl, ethyl, aminoethyl, dimethylaminoethyl, pyridylethyl, piperidinyl, pyrrolidinylethyl, piperazinylethyl and morpholinylethyl.

Other embodiments of the invention include compounds of formula (I) wherein at least one of R₅ and R₇ is selected from the group consisting of substituted and unsubstituted aryl, heteroaryl and biaryl. In some embodiments, at least one of R₅ and R₇ is a substituted or unsubstituted moiety of the formula (III): 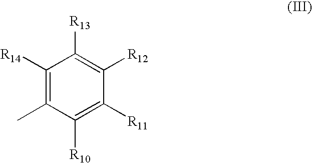
wherein R₁₀, R₁₁, R₁₂, R₁₃, and R₁₄ are independently selected from the group consisting of hydrogen, nitro, amino, cyano, halo, thioamido, carboxyl, hydroxy, and optionally substituted loweralkyl, loweralkoxy, loweralkoxyalkyl, haloloweralkyl, haloloweralkoxy, aminoalkyl, alkylamino, alkylthio, alkylcarbonylamino, aralkylcarbonylamino, heteroaralkylcarbonylamino, arylcarbonylamino, heteroarylcarbonylamino aminocarbonyl, loweralkylaminocarbonyl, aminoaralkyl, loweralkylaminoalkyl, aryl, heteroaryl, cycloheteroalkyl, aralkyl, alkylcarbonyloxy, arylcarbonyloxy, aralkylcarbonyloxy, arylcarbonyloxyalkyl, alkylcarbonyloxyalkyl, heteroarylcarbonyloxyalkyl, aralkycarbonyloxyalkyl, and heteroaralkcarbonyloxyalkyl.

In some embodiments, the invention provides compounds wherein R₁₀, R₁₁, R₁₃, and R₁₄ are hydrogen and R₁₂ is selected from the group consisting of halo, loweralkyl, hydroxy, loweralkoxy, haloloweralkyl, aminocarbonyl, alkylaminocarbonyl and cyano; R₁₁, R₁₃, and R₁₄ are hydrogen and R₁₀ and R₁₂ are independently selected from the group consisting of halo, loweralkyl, hydroxy, loweralkoxy, haloloweralkyl and cyano; R₁₀, R₁₁, R₁₃, and R₁₄ are hydrogen and R₁₂ is heteroaryl; R₁₀, R₁₁, R₁₃, and R₁₄ are hydrogen and R₁₂ is a heterocycloalkyl; and wherein at least one of R₁₀, R₁₁, R₁₂, R₁₃, and R₁₄ are halo and the remainder of R₁₀, R₁₁, R₁₂, R₁₃, and R₁₄ are hydrogen. Preferably, at least one of R₅ and R₇ is selected from the group consisting of dichlorophenyl, difluorophenyl, trifluoromethylphenyl, chlorofluorophenyl, bromochlorophenyl, ethylphenyl, methylchlorophenyl, imidazolylphenyl, cyanophenyl, morphlinophenyl and cyanochlorophenyl.

In other representative embodiments of the invention, R₆ in formula (I) may be substituted alkyl, such as, for example, aralkyl, hydroxyalkyl, aminoalkyl, aminoaralkyl, carbonylaminoalkyl, alkylcarbonylaminoalkyl, arylcarbonylaminoalkyl, aralkylcarbonylaminoalkyl, aminoalkoxyalkyl and arylaminoalkyl; substituted amino such as alkylamino, alkylcarbonylamino, alkoxycarbonylamino, arylalkylamino, arylcarbonylamino, alkylthiocarbonylamino, arylsulfonylamino, heteroarylamino alkylcarbonylamino, arylcarbonylamino, heteroarylcarbonylamino, aralkylcarbonylamino, and heteroaralkylcarbonylamino; or substituted carbonyl such as unsubstituted or substituted aminocarbonyl, alkyloxycarbonyl, aryloxycarbonyl, aralkyloxycarbonyl and alkylaminoalkyloxycarbonyl. In other embodiments, R₆ may be selected from the group consisting of amidino, guanidino, cycloimido, heterocycloimido, cycloamido, heterocycloamido, cyclothioamido and heterocycloloweralkyl. In yet other embodiments, R₆ may be aryl or heteroaryl, such as, for example, substituted or unsubstituted pyridyl, pyrimidinyl, thiazolyl, indolyl, imidazolyl, oxadiazolyl, tetrazolyl, pyrazinyl, triazolyl, thienyl, furanyl, quinolinyl, pyrrolyopyridyl, benzothiazolyl, benzopyridyl, benzotriazolyl, and benzimidazolyl.

As used herein, representative heterocyclo groups include, for example, those shown below (where the point of attachment of the substituent group, and the other substituent groups shown below, is through the upper left-hand bond). These heterocyclo groups can be further substituted and may be attached at various positions as will be apparent to those having skill in the organic and medicinal chemistry arts in conjunction with the disclosure herein.

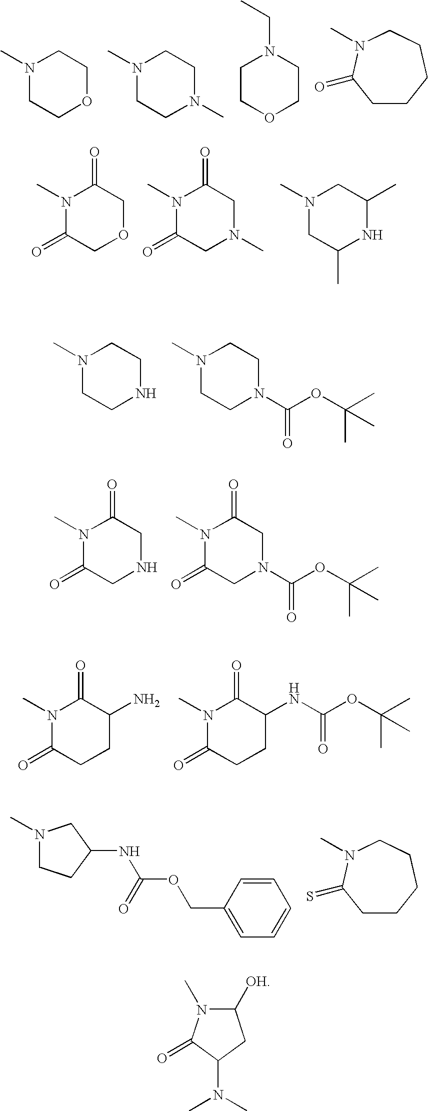

Representative heteroaryl groups include, for example, those shown below. These heteroaryl groups can be further substituted and may be attached at various positions as will be apparent to those having skill in the organic and medicinal chemistry arts in conjunction with the disclosure herein.

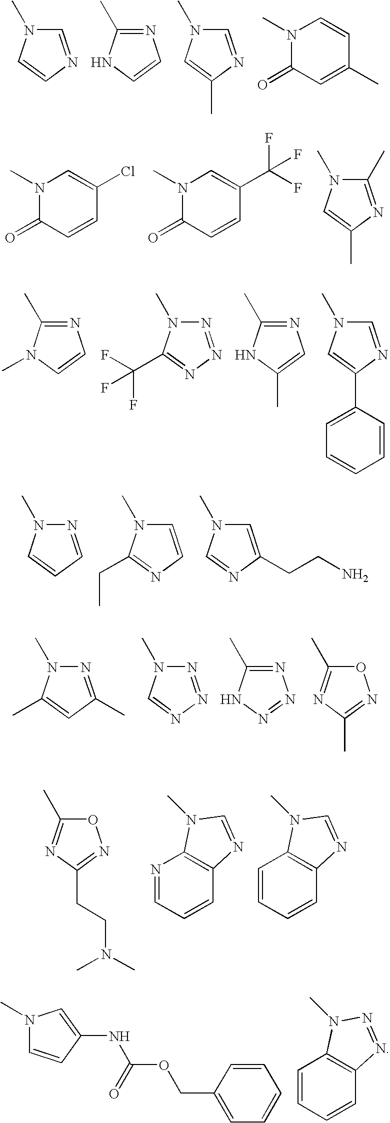

Representative cycloimido and heterocycloimido groups include, for example, those shown below. These cycloimido and heterocycloimido can be further substituted and may be attached at various positions as will be apparent to those having skill in the organic and medicinal chemistry arts in conjunction with the disclosure herein.

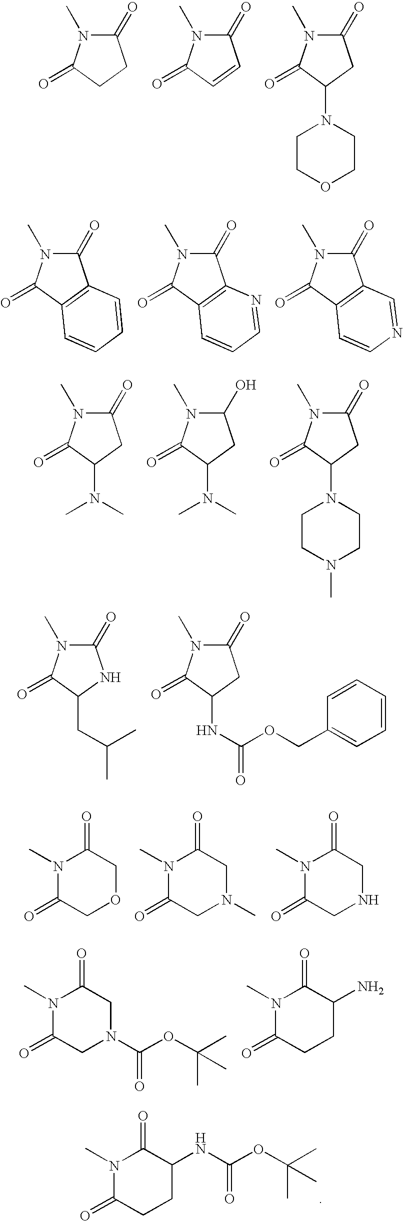

Representative substituted amidino and heterocycloamidino groups include, for example, those shown below. These amidino and heterocycloamidino groups can be further substituted as will be apparent to those having skill in the organic and medicinal chemistry arts in conjunction with the disclosure herein.

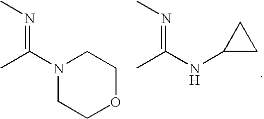

Representative substituted alkylcarbonylamino, alkyloxycarbonylamino, aminoalkyloxycarbonylamino, and arylcarbonylamino groups include, for example, those shown below. These groups can be further substituted as will be apparent to those having skill in the organic and medicinal chemistry arts in conjunction with the disclosure herein.

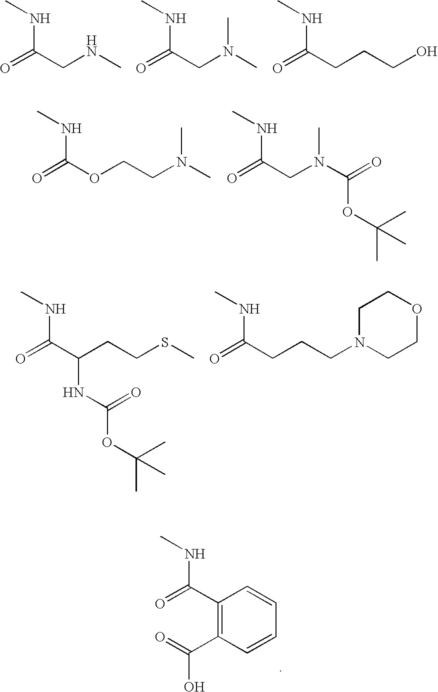

Representative substituted aminocarbonyl groups include, for example, those shown below. These can heterocyclo groups be further substituted as will be apparent to those having skill in the organic and medicinal chemistry arts in conjunction with the disclosure herein.

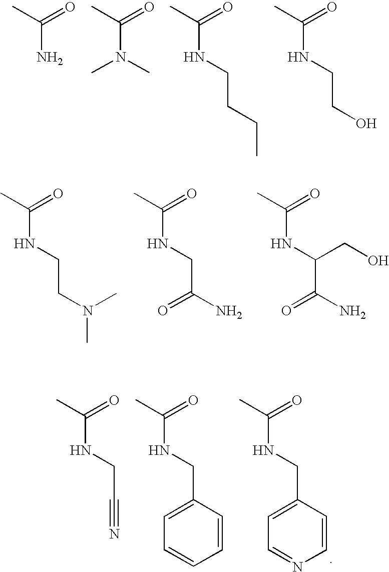

Representative substituted alkoxycarbonyl groups include, for example, those shown below. These alkoxycarbonyl groups can be further substituted as will be apparent to those having skill in the organic and medicinal chemistry arts in conjunction with the disclosure herein.

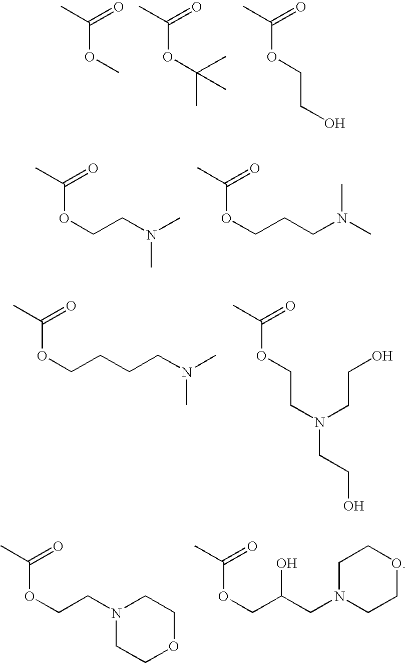

In some embodiments, compounds of the invention include compounds having the structure: 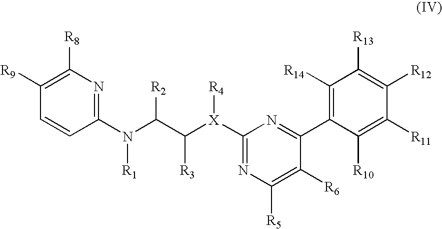
wherein X, R₁-R₆, and R₈-R₁₄ have the meanings described above, and the pharmaceutically acceptable salts thereof. Presently preferred, representative compounds of this group include, for example, [4-(4-imidazolylphenyl)pyrimidin-2-yl]{2-[(5-nitro(2-pyridyl))amino]ethyl}amine, 4-[5-imidazolyl-2-({2-[(5-nitro(2-pyridyl))amino]ethyl}amino)pyrimidin-4-yl]benzenecarbonitrile, 4-[2-({2-[(6-amino-5-nitro(2-pyridyl))amino]ethyl}amino)-5-imidazolylpyrimidin-4-yl]benzenecarbonitrile, [4-(2,4-dichlorophenyl)-5-imidazolylpyrimidin-2-yl]{2-[(5-nitro(2-pyridyl))amino]ethyl}amine, 4-[2-({2-[(5-nitro-2-pyridyl)amino]ethyl}amino)-7a-hydro-1,2,4-triazolo[1,5-a]pyrimidin-7-yl]benzenecarbonitrile, {2-[(6-amino-5-nitro-(2-pyridyl))amino]ethyl}[4-(2,4-dichlorophenyl)-5-imidazolylpyrimidin-2-yl]amine, [4-(2,4-dichlorophenyl)-5-imidazol-2-ylpyrimidin-2-yl]{2-[(5-nitro(2-pyridyl))amino]ethyl}amine, 6-[(2-{[4-(2,4-dichlorophenyl)-5-imidazolylpyrimidin-2-yl]amino}ethyl)amino]pyridine-3-carbonitrile, [5-benzotriazolyl-4-(2,4-dichlorophenyl)pyrimidin-2-yl]{2-[(5-nitro(2-pyridyl))amino]ethyl}amine, [2-({2-[(6-amino-5-nitro(2-pyridyl))amino]ethyl}amino)-4-(2,4-dichlorophenyl)pyrimidin-5-yl]methan-1-ol, [4-(2,4-dichlorophenyl)-2-({2-[(5-nitro(2-pyridyl))amino]ethyl}amino)pyrimidin-5-yl]methan-1-ol, 2-[2-({2-[(6-amino-5-nitro(2-pyridyl))amino]ethyl}amino)-4-(2,4-dichlorophenyl)pyrimidin-5-yl]isoindoline-1,3-dione, [5-amino-4-(2,4-dichlorophenyl)pyrimidin-2-yl]{2-[(6-amino-5-nitro(2-pyridyl))amino]ethyl}amine, {2-[(6-amino-5-nitro(2-pyridyl))amino]ethyl}[4-(2,4-dichlorophenyl)-5-morpholin-4-ylpyrimidin-2-yl]amine, {2-[(6-amino-5-nitro(2-pyridyl))amino]ethyl}{4-(2,4-dichlorophenyl)-5-[5-(trifluoromethyl)(1,2,3,4-tetraazolyl)]pyrimidin-2-yl}amine, 1-[2-({2-[(6-amino-5-nitro(2-pyridyl))amino]-ethyl}amino)-4-(2,4-dichlorophenyl)pyrimidin-5-yl]pyrrolidine-2,5-dione, [4-(2,4-dichlorophenyl)-5-pyrazolylpyrimidin-2-yl]{2-[(5-nitro(2-pyridyl))amino]ethyl}-amine, [4-(2,4-dichlorophenyl)-5-(4-methylimidazolyl)pyrimidin-2-yl]{2-[(5-nitro(2-pyridyl))amino]ethyl}amine, [4-(2,4-dichlorophenyl)-5-(2,4-dimethylimidazolyl)pyrimidin-2-yl]{2-[(5-nitro(2-pyridyl))amino]ethyl}amine, 6-[(2-{[4-(2,4-dichlorophenyl)-5-imidazol-2-ylpyrimidin-2-yl]amino}ethyl)amino]pyridine-3-carbonitrile, {2-[(6-amino-5-nitro(2-pyridyl))amino]ethyl}[4-(2,4-dichlorophenyl)-5-(morpholin-4-ylmethyl)pyrimidin-2-yl]amine, {2-[(6-amino-5-nitro(2-pyridyl))amino]ethyl}[4-(2,4-dichlorophenyl)-5-piperazinylpyrimidin-2-yl]amine, {2-[(6-amino-5-nitro(2-pyridyl))amino]ethyl}[4-(4-ethylphenyl)-5-imidazolylpyrimidin-2-yl]amine, 1-[4-(2,4-dichlorophenyl)-2-({2-[(5-nitro(2-pyridyl))amino]ethyl}amino)pyrimidin-5-yl]hydropyridin-2-one, [5-benzimidazolyl-4-(2,4-dichlorophenyl)pyrimidin-2-yl]{2-[(5-nitro(2-pyridyl))amino]ethyl}amine, {2-[(6-amino-5-nitro(2-pyridyl))amino]ethyl}[4-(2,4-dichlorophenyl)-5-imidazolylpyrimidin-2-yl]methyl-amine, {2-[(6-amino-5-nitro(2-pyridyl))amino]ethyl}[4-(2,4-dichlorophenyl)-5-(4-pyridyl)pyrimidin-2-yl]amine, {2-[(6-amino-5-nitro(2-pyridyl))amino]ethyl}[4-(2,4-dichlorophenyl)-5-(4-methylpiperazinyl)pyrimidin-2-yl]amine, [4-(2,4-dichlorophenyl)-5-(2-methylimidazolyl)pyrimidin-2-yl]{2-[(5-nitro(2-pyridyl))amino]ethyl}-amine, {2-[(6-amino-5-nitro(2-pyridyl))amino]ethyl}[4-(2,4-dichlorophenyl)-5-(2-methylimidazolyl)pyrimidin-2-yl]amine, {2-[(6-amino-5-nitro(2-pyridyl))amino]-ethyl}[4-(2,4-dichlorophenyl)-5-(4-phenylimidazolyl)pyrimidin-2-yl]amine, {2-[(6-amino-5-nitro(2-pyridyl))amino]ethyl}[4-(2,4-dichlorophenyl)-5-(2,4-dimethylimidazolyl)pyrimidin-2-yl]amine, [4-(2,4-dichlorophenyl)-5-imidazol-2-ylpyrimidin-2-yl](2-{[5-(trifluoromethyl)(2-pyridyl)]amino}ethyl)amine, [4-(2,4-dichlorophenyl)-5-piperazinylpyrimidin-2-yl]{2-[(5-nitro(2-pyridyl))amino]ethyl}amine, [4-(2,4-dichlorophenyl)-5-imidazolylpyrimidin-2-yl][2-(dimethylamino)ethyl]{2-[(5-nitro(2-pyridyl))amino]ethyl}amine, 1-[2-({2-[(6-amino-5-nitro(2-pyridyl))amino]ethyl}-amino)-4-(2,4-dichlorophenyl)pyrimidin-5-yl]-4-methylpiperazine-2,6-dione, [4-(2,4-dichlorophenyl)-5-(1-methylimidazol-2-yl)pyrimidin-2-yl]{2-[(5-nitro(2-pyridyl))amino]ethyl}amine, 1-[2-({2-[(6-amino-5-nitro(2-pyridyl))amino]ethyl}amino)-4-(2,4-dichlorophenyl)pyrimidin-5-yl]-3-morpholin-4-ylpyrrolidine-2,5-dione, 1-[4-(2,4-dichlorophenyl)-2-({2-[(5-nitro(2-pyridyl))amino]ethyl}amino)pyrimidin-5-yl]-4-methylpiperazine-2,6-dione, 1-[2-({2-[(6-amino-5-nitro(2-pyridyl))amino]ethyl}-amino)-4-(2,4-dichlorophenyl)pyrimidin-5-yl]-3-(dimethylamino)pyrrolidine-2,5-dione, {5-imidazol-2-yl-4-[4-(trifluoromethyl)phenyl]pyrimidin-2-yl}{2-[(5-nitro(2-pyridyl))amino]ethyl}amine, {2-[(6-amino-5-nitro(2-pyridyl))amino]ethyl}[4-(2,4-dichlorophenyl)-5-(1-methylimidazol-2-yl)pyrimidin-2-yl]amine, [4-(2,4-dichlorophenyl)-5-(4-methylpiperazinyl)pyrimidin-2-yl]{2-[(5-nitro(2-pyridyl))amino]ethyl}amine, [4-(2,4-dichlorophenyl)-5-(morpholin-4-ylmethyl)pyrimidin-2-yl]{2-[(5-nitro(2-pyridyl))amino]ethyl}amine, [4-(2,4-dichlorophenyl)-5-(4-methylimidazol-2-yl)pyrimidin-2-yl]{2-[(5-nitro(2-pyridyl))amino]-ethyl}amine, {2-[(6-amino-5-nitro(2-pyridyl))amino]ethyl}[4-(2,4-dichlorophenyl)-5-(4-methylimidazol-2-yl)pyrimidin-2-yl]amine, {2-[(6-amino-5-nitro(2-pyridyl))amino]ethyl}[4-(2-chlorophenyl)-5-imidazol-2-ylpyrimidin-2-yl]amine, [4-(2-chloro-4-fluorophenyl)-5-imidazol-2-ylpyrimidin-2-yl]{2-[(5-nitro(2-pyridyl))amino]ethyl}amine, [4-(2,4-dichlorophenyl)-5-imidazolylpyrimidin-2-yl]{2-[(5-nitro(2-pyridyl))amino]ethyl}(2-pyrrolidinylethyl)amine, [4-(2,4-dichlorophenyl)-5-imidazolylpyrimidin-2-yl](2-morpholin-4-ylethyl){2-[(5-nitro(2-pyridyl))amino]ethyl}amine, 6-[(2-{[4-(2,4-dichlorophenyl)-5-(4-methylimidazol-2-yl)pyrimidin-2-yl]amino}ethyl)amino]pyridine-3-carbonitrile, {2-[(6-amino-5-nitro(2-pyridyl))amino]ethyl}[4-(2-chloro-4-fluorophenyl)-5-imidazol-2-ylpyrimidin-2-yl]amine, [4-(4-ethylphenyl)-5-imidazol-2-ylpyrimidin-2-yl]{2-[(5-nitro(2-pyridyl))amino]ethyl}amine, [5-((1E)-1-aza-2-morpholin-4-ylprop-1-enyl)-4-(2,4-dichlorophenyl)pyrimidin-2-yl]{2-[(6-amino-5-nitro(2-pyridyl))amino]ethyl}amine, N-[4-(2,4-dichlorophenyl)-2-({2-[(5-nitro(2-pyridyl))amino]ethyl}amino)pyrimidin-5-yl]acetamide, [4-(2,4-dichlorophenyl)-5-imidazol-2-ylpyrimidin-2-yl]{2-[(6-methoxy-5-nitro(2-pyridyl))amino]ethyl}amine, 6-[(2-{[4-(2,4-dichlorophenyl)-5-imidazolylpyrimidin-2-yl]methylamino}ethyl)amino]pyridine-3-carbonitrile, 6-[(2-{[4-(2,4-dichlorophenyl)-5-imidazol-2-ylpyrimidin-2yl]methylamino}ethyl)amino]pyridine-3-carbonitrile, [4-(2,4-dichlorophenyl)-5-imidazol-2-ylpyrimidin-2-yl]methyl{2-[(5-nitro(2-pyridyl))amino]ethyl}amine, 6-[(2-{[4-(2-chloro-4-fluoro-phenyl)-5-imidazol-2-ylpyrimidin-2-yl]amino}ethyl)amino]pyridine-3-carbonitrile, [4-(4-chlorophenyl)-5-imidazol-2-ylpyrimidin-2-yl]{2-[(5-nitro(2-pyridyl))amino]ethyl}amine, {2-[(6-amino-5-nitro(2-pyridyl))amino]ethyl}[4-(4-chloro-2-methylphenyl)-5-imidazol-2-ylpyrimidin-2-yl]amine, {2-[(6-amino-5-nitro(2-pyridyl))amino]ethyl}[4-(4-bromo-2-chlorophenyl)-5-imidazol-2-ylpyrimidin-2-yl]amine, 6-[(2-{[4-(4-bromo-2-chlorophenyl)-5-imidazol-2-ylpyrimidin-2-yl]amino}ethyl)amino]pyridine-3-carbonitrile, 6-[2-({2-[(6-amino-5-nitro(2-pyridyl))amino]ethyl}amino)-4-(2,4-dichlorophenyl)pyrimidin-5-yl]-3-pyrrolino[3,4-b]pyridine-5,7-dione, N-[2-({2-[(6-amino-5-nitro(2-pyridyl))amino]ethyl}amino)-4-(2,4-dichlorophenyl)pyrimidin-5-yl]-2-(methylamino)acetamide, {2-[(6-amino-5-nitro(2-pyridyl))amino]ethyl}[4-(4-bromo-2-chlorophenyl)-5-(4-methylimidazol-2-yl)pyrimidin-2-yl]amine, 6-[(2-{[4-(4-bromo-2-chlorophenyl)-5-(4-methylimidazol-2-yl)pyrimidin-2-yl]amino}ethyl)amino]pyridine-3-carbonitrile, {2-[(6-amino-5-nitro(2-pyridyl))amino]ethyl}[4-(2-chloro-4-fluorophenyl)-5-(4-methylimidazol-2-yl)pyrimidin-2-yl]amine, and 6-[(2-{[4-(2,4-dichlorophenyl)-5-(5-chloro-2-oxohydropyridyl)pyrimidin-2-yl]amino}ethyl)amino]pyridine-3-carbonitrile.

In other embodiments, the invention provides compounds having the structure: 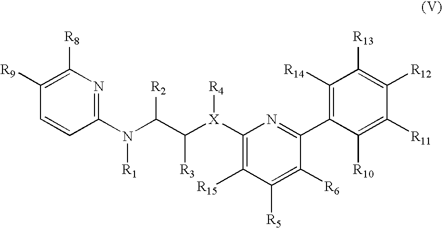
wherein X, R₁-R₆, and R₈-R₁₄ have the meanings described above, and R₁₅ is selected from the group consisting of hydrogen, nitro, cyano, amino, alkyl, halo, haloloweralkyl, alkyloxycarbonyl, aminocarbonyl, alkylsulfonyl and arylsulfonyl, and the pharmaceutically acceptable salts thereof. Presently preferred, representative compounds of this group include, for example, [6-(2,4-dichlorophenyl)-5-imidazolyl(2-pyridyl)]{2-[(5-nitro(2-pyridyl))amino]ethyl}amine, {2-[(6-amino-5-nitro(2-pyridyl))amino]ethyl}[6-(2,4-dichlorophenyl)-5-imidazolyl(2-pyridyl)]amine, 6-[(2-{[6-(2,4-dichlorophenyl)-5-imidazolyl-2-pyridyl]amino}ethyl)amino]pyridine-3-carbonitrile, {2-[(6-amino-5-nitro(2-pyridyl))amino]ethyl}[6-(2,4-dichlorophenyl)-5-nitro(2-pyridyl)]amine, {2-[(6-amino-5-nitro(2-pyridyl))amino]ethyl}[6-(2,4-dichlorophenyl)-5-(4-methylimidazolyl)(2-pyridyl)]amine, 6-[(2-{[6-(2,4-dichlorophenyl)-5-(4-methylimidazolyl)-2-pyridyl]amino}ethyl)amino]pyridine-3-carbonitrile, and [4-(4-bromo-2-chlorophenyl)-5-imidazol-2-ylpyrimidin-2-yl]{2-[(5-nitro(2-pyridyl))amino]ethyl}amine.

A preferred compound of formula (I) is compound (VI) 6-[(2-{[4-(2,4-dichlorophenyl)-5-(4-methylimidazol-2-yl)pyrimidin-2-yl]amino}ethyl)amino]pyridine-3-carbonitrile having the following formula:

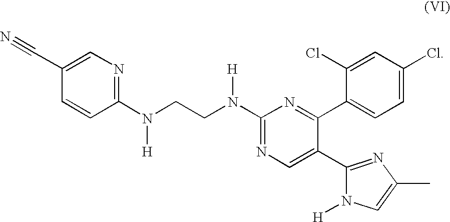

In another aspect, the invention provides compositions comprising an amount of a compound of formula I effective to modulate GSK3 activity in a human or animal subject when administered thereto, together with a pharmaceutically acceptable carrier.

In yet other embodiments, the invention provides methods of inhibiting GSK3 activity in a human or animal subject, comprising administering to the human or animal subject a GSK3 inhibitory amount of a compound of formula (I).

The present invention further provides methods of treating human or animal subjects suffering from GSK3-mediated disorder in a human or animal subject, comprising administering to the human or animal subject a therapeutically effective amount of a compound of formula (I) above, either alone or in combination with other therapeutically active agents.

As used above and elsewhere herein the following terms have the meanings defined below:

“Glycogen synthase kinase 3” and “GSK3” are used interchangeably herein to refer to any protein having more than 60% sequence homology to the amino acids between positions 56 and 340 of the human GSK3 beta amino acid sequence (Genbank Accession No. L33801). To determine the percent homology of two amino acid sequences or of two nucleic acids, the sequences are aligned for optimal comparison purposes (e.g., gaps can be introduced in the sequence of one polypeptide or nucleic acid for optimal alignment with the other polypeptide or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100). GSK3 was originally identified by its phosphorylation of glycogen synthase as described in Woodgett et. al., Trends Biochem. Sci., 16:177-81 (1991), incorporated herein by reference. By inhibiting GSK3 kinase activity, activities downstream of GSK3 activity may be inhibited, or, alternatively, stimulated. For example, when GSK3 activity is inhibited, glycogen synthase may be activated, resulting in increased glycogen production. GSK3 is also known to act as a kinase in a variety of other contexts, including, for example, phosphorylation of c-jun, β-catenin, and tau protein. It is understood that inhibition of GSK3 kinase activity can lead to a variety of effects in a variety of biological contexts. The invention, however, is not limited by any theories of mechanism as to how the invention works.

“GSK3 inhibitor” is used herein to refer to a compound that exhibits an IC₅₀ with respect to GSK3 of no more than about 100 μM and more typically not more than about 50 μM, as measured in the cell-free assay for GSK3 inhibitory activity described generally hereinbelow. “IC₅₀” is that concentration of inhibitor which reduces the activity of an enzyme (e.g., GSK3) to half-maximal level. Representative compounds of the present invention have been discovered to exhibit inhibitory activity against GSK3. Compounds of the present invention preferably exhibit an IC₅₀ with respect to GSK3 of no more than about 10 μM, more preferably, no more than about 5 μM, even more preferably not more than about 1 μM, and most preferably, not more than about 200 nM, as measured in the cell-free GSK3 kinase assay.

“Optionally substituted” refers to the replacement of hydrogen with a monovalent or divalent radical. Suitable substitution groups include, for example, hydroxyl, nitro, amino, imino, cyano, halo, thio, thioamido, amidino, imidino, oxo, oxamidino, methoxamidino, imidino, guanidino, sulfonamido, carboxyl, formyl, loweralkyl, haloloweralkyl, loweralkoxy, haloloweralkoxy, loweralkoxyalkyl, alkylcarbonyl, arylcarbonyl, aralkylcarbonyl, heteroarylcarbonyl, heteroaralkylcarbonyl, alkylthio, aminoalkyl, cyanoalkyl, and the like.

The substitution group can itself be substituted. The group substituted onto the substitution group can be carboxyl, halo; nitro, amino, cyano, hydroxyl, loweralkyl, loweralkoxy, aminocarbonyl, —SR, thioamido, —SO₃H, —SO₂R or cycloalkyl, where R is typically hydrogen, hydroxyl or loweralkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substitutents can be straight chain, branched or cyclic arrangements of covalently bonded carbon or heteroatoms.

“Loweralkyl” as used herein refers to branched or straight chain alkyl groups comprising one to ten carbon atoms that are unsubstituted or substituted, e.g., with one or more halogen, hydroxyl or other groups, including, e.g., methyl, ethyl, propyl, isopropyl, n-butyl, t-butyl, neopentyl, trifluoromethyl, pentafluoroethyl and the like.

“Alkylenyl” refers to a divalent straight chain or branched chain saturated aliphatic radical having from 1 to 20 carbon atoms. Typical alkylenyl groups employed in compounds of the present invention are loweralkylenyl groups that have from 1 to about 6 carbon atoms in their backbone. “Alkenyl” refers herein to straight chain, branched, or cyclic radicals having one or more double bonds and from 2 to 20 carbon atoms. “Alkynyl” refers herein to straight chain, branched, or cyclic radicals having one or more triple bonds and from 2 to 20 carbon atoms.

“Loweralkoxy” as used herein refers to RO— wherein R is loweralkyl. Representative examples of loweralkoxy groups include methoxy, ethoxy, t-butoxy, trifluoromethoxy and the like.

“Cycloalkyl” refers to a mono- or polycyclic, heterocyclic or carbocyclic alkyl substituent. Typical cycloalkyl substituents have from 3 to 8 backbone (i.e., ring) atoms in which each backbone atom is either carbon or a heteroatom. The term “heterocycloalkyl” refers herein to cycloalkyl substituents that have from 1 to 5, and more typically from 1 to 4 heteroatoms in the ring structure. Suitable heteroatoms employed in compounds of the present invention are nitrogen, oxygen, and sulfur. Representative heterocycloalkyl moieties include, for example, morpholino, piperazinyl, piperadinyl and the like. Carbocycloalkyl groups are cycloalkyl groups in which all ring atoms are carbon. When used in connection with cycloalkyl substituents, the term “polycyclic” refers herein to fused and non-fused alkyl cyclic structures.

“Halo” refers herein to a halogen radical, such as fluorine, chlorine, bromine or iodine. “Haloalkyl” refers to an alkyl radical substituted with one or more halogen atoms. The term “haloloweralkyl” refers to a loweralkyl radical substituted with one or more halogen atoms. The term “haloalkoxy” refers to an alkoxy radical substituted with one or more halogen atoms. The term “haloloweralkoxy” refers to a loweralkoxy radical substituted with one or more halogen atoms.

“Aryl” refers to monocyclic and polycyclic aromatic groups having from 3 to 14 backbone carbon or hetero atoms, and includes both carbocyclic aryl groups and heterocyclic aryl groups. Carbocyclic aryl groups are aryl groups in which all ring atoms in the aromatic ring are carbon. The term “heteroaryl” refers herein to aryl groups having from 1 to 4 heteroatoms as ring atoms in an aromatic ring with the remainder of the ring atoms being carbon atoms. When used in connection with aryl substituents, the term “polycyclic” refers herein to fused and non-fused cyclic structures in which at least one cyclic structure is aromatic, such as, for example, benzodioxozolo (which has a heterocyclic structure fused to a phenyl group, i.e. 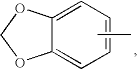
naphthyl, and the like. Exemplary aryl moieties employed as substituents in compounds of the present invention include phenyl, pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, and the like.

“Aralkyl” refers to an alkyl group substituted with an aryl group. Typically, aralkyl groups employed in compounds of the present invention have from 1 to 6 carbon atoms incorporated within the alkyl portion of the aralkyl group. Suitable aralkyl groups employed in compounds of the present invention include, for example, benzyl, picolyl, and the like.

“Amino” refers herein to the group —NH₂. The term “alkylamino” refers herein to the group —NRR′ where R and R′ are each independently selected from hydrogen or a lower alkyl. The term “arylamino” refers herein to the group —NRR′ where R is aryl and R′ is hydrogen, a lower alkyl, or an aryl. The term “aralkylamino” refers herein to the group —NRR′ where R is a lower aralkyl and R′ is hydrogen, a loweralkyl, an aryl, or a loweraralkyl.

The term “arylcycloalkylamino” refers herein to the group, aryl-cycloalkyl-NH—, where cycloalkyl is a divalent cycloalkyl group. Typically, cycloalkyl has from 3 to 6 backbone atoms, of which, optionally 1 to about 4 are heteroatoms. The term “aminoalkyl” refers to an alkyl group that is terminally substituted with an amino group.

The term “alkoxyalkyl” refers to the group -alk₁-O-alk₂ where alk₁ is alkylenyl or alkenyl, and alk₂ is alkyl or alkenyl. The term “loweralkoxyalkyl” refers to an alkoxyalkyl where alk₁ is loweralkylenyl or loweralkenyl, and alk₂ is loweralkyl or loweralkenyl. The term “aryloxyalkyl” refers to the group -alkylenyl-O-aryl. The term “aralkoxyalkyl” refers to the group -alkylenyl-O-aralkyl, where aralkyl is a loweraralkyl.

The term “alkoxyalkylamino” refers herein to the group —NR— (alkoxylalkyl), where R is typically hydrogen, loweraralkyl, or loweralkyl. The term “aminoloweralkoxyalkyl” refers herein to an aminoalkoxyalkyl in which the alkoxyalkyl is a loweralkoxyalkyl.

The term “aminocarbonyl” refers herein to the group —C(O)—NH₂. “Substituted aminocarbonyl” refers herein to the group —C(O)—NRR′ where R is loweralkyl and R′ is hydrogen or a loweralkyl. The term “arylaminocarbonyl” refers herein to the group —C(O)—NRR′ where R is an aryl and R′ is hydrogen, loweralkyl or aryl. “aralkylaminocarbonyl” refers herein to the group —C(O)—NRR′ where R is loweraralkyl and R′ is hydrogen, loweralkyl, aryl, or loweraralkyl.

“Aminosulfonyl” refers herein to the group —S(O)₂—NH₂. “Substituted aminosulfonyl” refers herein to the group —S(O)₂—NRR′ where R is loweralkyl and R′ is hydrogen or a loweralkyl. The term “aralkylaminosulfonlyaryl” refers herein to the group -aryl-S(O)₂—NH-aralkyl, where the aralkyl is loweraralkyl.

“Carbonyl” refers to the divalent group —C(O)—.

“Carbonyloxy” refers generally to the group —C(O)—O—. Such groups include esters, —C(O)—O—R, where R is loweralkyl, cycloalkyl, aryl, or loweraralkyl. The term “carbonyloxycycloalkyl” refers generally herein to both an “carbonyloxycarbocycloalkyl” and an “carbonyloxyheterocycloalkyl”, i.e., where R is a carbocycloalkyl or heterocycloalkyl, respectively. The term “arylcarbonyloxy” refers herein to the group —C(O)—O-aryl, where aryl is a mono- or polycyclic, carbocycloaryl or heterocycloaryl. The term “aralkylcarbonyloxy” refers herein to the group —C(O)—O-aralkyl, where the aralkyl is loweraralkyl.

The term “sulfonyl” refers herein to the group —SO₂—. “Alkylsulfonyl” refers to a substituted sulfonyl of the structure —SO₂R— in which R is alkyl. Alkylsulfonyl groups employed in compounds of the present invention are typically loweralkylsulfonyl groups having from 1 to 6 carbon atoms in its backbone structure. Thus, typical alkylsulfonyl groups employed in compounds of the present invention include, for example, methylsulfonyl (i.e., where R is methyl), ethylsulfonyl (i.e., where R is ethyl), propylsulfonyl (i.e., where R is propyl), and the like. The term “arylsulfonyl” refers herein to the group —SO₂-aryl. The term “aralkylsulfonyl” refers herein to the group —SO₂-aralkyl, in which the aralkyl is loweraralkyl. The term “sulfonamido” refers herein to —SO₂NH₂.

As used herein, the term “carbonylamino” refers to the divalent group —NH—C(O)— in which the hydrogen atom of the amide nitrogen of the carbonylamino group can be replaced a loweralkyl, aryl, or loweraralkyl group. Such groups include moieties such as carbamate esters (—NH—C(O)—O—R) and amides —NH—C(O)—O—R, where R is a straight or branched chain loweralkyl, cycloalkyl, or aryl or loweraralkyl. The term “loweralkylcarbonylamino” refers to alkylcarbonylamino where R is a loweralkyl having from 1 to about 6 carbon atoms in its backbone structure. The term “arylcarbonylamino” refers to group —NH—C(O)—R where R is an aryl. Similarly, the term “aralkylcarbonylamino ” refers to carbonylamino where R is a lower aralkyl.

As used herein, the term “guanidino” or “guanidyl” refers to moieties derived from guanidine, H₂N—C(═NH)—NH₂. Such moieties include those bonded at the nitrogen atom carrying the formal double bond (the “2”-position of the guanidine, e.g., diaminomethyleneamino, (H₂N)₂C═NH—) and those bonded at either of the nitrogen atoms carrying a formal single bond (the “1-” and/or “3”-positions of the guandine, e.g., H₂N—C(═NH)—NH—). The hydrogen atoms at any of the nitrogens can be replaced with a suitable substituent, such as loweralkyl, aryl, or loweraralkyl.

As used herein, the term “amidino” refers to the moieties R—C(═N)—NR′— (the radical being at the “N¹” nitrogen) and R(NR′)C═N— (the radical being at the “N²” nitrogen), where R and R′ can be hydrogen, loweralkyl, aryl, or loweraralkyl.

The term “bone loss” refers any condition in which there is loss of bone mineral density.

The term “anti-resorption agent” refers to resorption inhibitors such as bisphosphonates, selective estrogen receptor modulators (SERMs), oestrogens, RANKL (receptor activator of nuclear factor NF-κB ligand) antagonists, αᵥβ₃ antagonists, scr inhibitors, cathepsin K inhibitors, and calcitonin.

The term “osteogenic promoting agent” refers to compounds and peptides that stimulate osteogenesis. Osteogenic promoting agents includes recombinant parathyroid hormones such as Teriparatide.

Compounds of the present invention can be readily synthesized using the methods described herein, or other methods, which are well known in the art. The compounds of the present invention can be synthesized according to the methods described in U.S. Pat. Nos. 6,417,185, 6,489,344, and PCT WO 99/65897 and WO 02/20495.

For example, the synthesis of pyrimidines having a wide variety of substituents is comprehensibly reviewed in D. J. Brown, “The Pyrimidines,” vol. 54, Wiley (1994), which is incorporated herein by reference. The compounds described herein were synthesized using both solution-phase and resin-based (i.e., solid-phase) techniques.

Pyrimidine based compounds of the present invention can be readily synthesized in solution by reaction of a carbonyl-containing derivative with N,N-dimethylformamide dimethyl acetal (DMFDMA). The intermediate enaminoketone that results is then reacted with a guanidine in the presence of an organic solvent and a suitable base such as sodium ethoxide, sodium methoxide, sodium hydroxide or cesium carbonate at various temperatures to give a pyrimidine. This method is generally described in Menozzi et. al., J. Heterocyclic Chem., 24:1669 (1987), P. Schenone et. al., J. Heterocyclic Chem., 27: 295 (1990), R. Paul et. al., J. Med. Chem., 36: 2716 (1993) and J. Zimmermann et. al., Arch. Pharm., 329: 371 (1996), all of which are incorporated herein by reference.

Carbonyl-containing starting reagents that are suitable for use in this reaction scheme include, for example, β-keto esters, alkyl aryl ketones, β-keto sulfones, α-nitro ketones, β-keto nitriles, desoxybenzoins, aryl heteroarylmethyl ketones, and the like. The carbonyl-containing starting reagents can either be purchased or synthesized using known methods.

For example, β-keto esters can be readily synthesized by reaction of an acid chloride or other activated carboxylic acid with potassium ethyl malonate in the presence of triethylamine in accordance with the method described in R. J. Clay et. al., Synthesis, 1992: 290 (1992), which is incorporated herein by reference. Alternatively, the desired β-keto ester can be synthesized by deprotonating an appropriate methyl ketone with a suitable base such as sodium hydride, followed by condensation with diethylcarbonate in accordance with the method described in Sircar et. al., J. Med. Chem., 28:1405 (1985), which is incorporated herein by reference.

Likewise, β-keto sulfones and α-nitro ketones can be prepared using known methods, such as those described in N. S. Simpkins, “Sulphones in Organic Synthesis,” Pergamon (1993) (α-keto sulfones) and M. Jung et. al., J. Org. Chem., 52:4570 (1987) (α-nitro ketones), both of which are incorporated herein by reference. β-keto nitriles can be readily prepared by reaction of an α-halo ketone with sodium or potassium cyanide.

When the substrate is a doubly activated carbonyl compound (e.g., β-keto ester, β-keto sulfone, β-keto nitrile, and the like) the first condensation is typically conducted with a small excess of DMFDMA in a solvent such as THF at 70-80° C. for several hours

When a mono-activated substrate such as a methyl ketone is involved, DMFDMA is often used as the solvent at a higher temperature (90-100° C.) for a longer period of time (e.g., overnight). After completion of the condensation reaction, the solvent and excess DMFDMA are removed in vacuo. The resulting solid or oil is dissolved in an appropriate solvent and heated with an equimolar amount of the guanidine and base.

When esters are formed, alkaline or acidic hydrolysis of the resulting pyrimidine yields the corresponding carboxylic acid. This acid can then be further coupled to various alcohols or amines to provide a variety of ester or amide derivatives.

Guanidines employed in the synthesis of invention compounds can be purchased or, alternatively, synthesized by reacting the corresponding amine with a guanidino transfer reagent, such as, for example, benzotriazole carboxamidinium 4-methylbenzenesulfonate. This guanidino transfer reagent is described in A. R. Katritzky et. al., 1995, Synthetic Communications, 25:1173 (1995), which is incorporated herein by reference. Thus, for example, benzotriazole carboxamidinium 4-methylbenzenesulfonate can be reacted in equimolar quantity with an amine and one equivalent of diisopropyl ethyl amine (DIEA) in acetonitrile at room temperature overnight to yield guanidinium 4-methylbenzenesulfonate upon addition of diethyl ether. Amines containing a nitrogen heterocyclic aryl can be prepared by nucleophilic substitution of a halo-substituted nitrogen heterocyclic aryl with an appropriate diamine, such as, for example, ethylenediamine or propylenediamine. These diamines are particularly suitable for use as reaction solvents at reaction temperatures in the range of about 25° C. to 125° C. The preparation of specialized amines is noted in the Examples provided herein.

Other known synthesis methods can be used to prepare compounds of the present invention. For example, 5-aryl 2-aminopyrimidine can be prepared by reacting a guanidine with a vinamidinium salt, in accordance with the method described in R. M. Wagner and C. Jutz, Chem. Berichte, p. 2975 (1971), which is incorporated herein by reference.

Similarly, 4-anilo-2-chloropyrimidine can be prepared by reacting aniline with 2,4-dichloropyrimidine. Likewise, an aniline can be treated with a 2,4-dichloropyrimidine to give the 4-anilo-2-chloropyrimidine. Further substitution with a second amine gives 2-amino-4-anilinopyrimidine.

In addition to solution-phase synthesis methods, solid-support (including resin-based) synthesis methods can also be used to synthesize compounds of the present invention, especially for parallel and combinatorial synthesis methodologies. For example, the synthesis of tetra-substituted pyrimidines may begin with the loading of an aromatic carboxylic acid aldehyde, such as, for example, 4-formyl benzoic acid, to the amino group of a suitable resin, such as, for example, Rink amide resin (Novabiochem, San Diego, Calif.) (“Resin Method A”) Knoevenagel condensation of a β-keto ester gives an unsaturated intermediate that can be condensed with 1H-pyrazole-1-carboxamidine hydrochloride (Aldrich) in the presence of a suitable base (e.g., potassium carbonate). The intermediate dihydropyrimidine can then be oxidized to the resin bound pyrimidine with 2,3-dichloro-5,6-dicyano-1,4-benzoquinone (DDQ) in benzene. Finally, substitution of the pyrazolo moiety by heating with an amine in 1-methylpyrrolidone (NMP) or other suitable solvent is followed by acidolytic cleavage to give the desired pyrimidine. This synthesis method can be used to generate pyrimidines with a substituent in the 4-position of the pyrimidine ring.

Resin Method B, can be used to synthesize pyrimidines in which the 6-position is unsubstituted. A hydroxymethyl-resin, such as commercially available Sasrin resin (Bachem Biosciences, King of Prussia, Pa.), is treated with triphenylphosphine dibromide in dichloromethane to convert the hydroxymethyl group on the resin to a bromomethyl group, as generally described in K. Ngu et. al., Tetrahedron Letters, 38: 973 (1997), which is incorporated herein by reference. The bromine is then displaced by reaction with a primary amine in NMP (at room temperature or 70-80° C.). The amine is then coupled with the appropriate aromatic compound containing an acetyl group. The coupling can be carried out with PyBOP® (Novabiochem, San Diego, Calif.), and 4-methylmorpholine in NMP.

Resin Method B can also be used to incorporate an amino acid residue into the resulting pyrimidine. For example, amino resin can be coupled to a 9-fluorenyl-methoxycarbonyl (FMOC)-protected amino acid using standard peptide synthesis conditions and methods. Further coupling with 4-acetylbenzoic acid followed by reaction with N,N-dimethylformamide dimethyl acetal and cyclization with a guanidine produces a pyrimidine derivative having an amino acid residue incorporated within it.

Pyrimidines having e.g., a carboxamidophenyl group at position 6 and hydrogen at position 5 can be prepared from an amino (i.e., —NH₂)-containing resin such as Rink amide resin (Novabiochem, San Diego, Calif.) (“Resin Method C”).

Compounds of the present invention can also be prepared according to Resin Method D, to produce 2,4-diaminopyrimidines. Resin-bound amine is reacted with a 2,4-dichloropyrimidine to give a resin-bound 6-amino-2-chloropyrimidine. The resin-bound amine can be derived from any suitable primary amine; however, anilines generally are not suitable. Displacement with a second amine and cleavage of the product from the resin gives a 2,4-diaminopyrimidine. For the second displacement, primary or secondary amines that may contain other functional groups, such as unprotected hydroxy groups, are suitable. The resulting dichloropyrimidine may be further substituted, for example, with an ester group at the 5-position. A 2,6-dichloropyridine can be used instead of 2,4-dichloropyrimidine to produce a 2,6-diaminopyridine.

Resin Method E can be used to produce a 2,6-diaminopyridine. The method is analogous to Resin Method D except that a 2,6-dichloropyridine is used as the electrophile and the final product is a 2,6-diaminopyridine.

Resin Method F can be used to synthesize 5-amino substituted compounds of the present invention. Resin-bound amine is reacted with a halomethyl aryl ketone. The resulting resin-bound aminomethyl ketone is then treated with DMFDMA (neat) followed by cyclization with a guanidine to give the 2,5-diamino-6-arylpyrimidine.

Resin Method G can be used to synthesize compounds of the present invention having a carboxyl group at the 5-position.

GSK3 inhibitor compounds of the present invention can be purified using known methods, such as, for example, chromatography, crystallization, and the like.

Compounds of the present invention preferably exhibit inhibitory activity that is relatively substantially selective with respect to GSK3, as compared to at least one other type of kinase. As used herein, the term “selective” refers to a relatively greater potency for inhibition against GSK3, as compared to at least one other type of kinase. Preferably, GSK3 inhibitors of the present invention are selective with respect to GSK3, as compared to at least two other types of kinases. Kinase activity assays for kinases other than GSK3 are generally known. See e.g., Havlicek et. al., J. Med. Chem., 40: 408-12 (1997), incorporated herein by reference. GSK3 selectivity can be quantitated according to the following: GSK3 selectivity=IC_{50 (other kinase)}÷IC_{50 (GSK3)}, where a GSK3 inhibitor is selective for GSK3 when IC_{50 (other kinase)}>IC_{50 (GSK3)}. Thus, an inhibitor that is selective for GSK3 exhibits a GSK3 selectivity of greater than 1-fold with respect to inhibition of a kinase other than GSK3. As used herein, the term “other kinase” refers to a kinase other than GSK3. Such selectivities are generally measured in cell-free assays.

Typically, GSK3 inhibitors of the present invention exhibit a selectivity of at least about 2-fold (i.e., IC_{50 (other kinase)}÷IC_{50 (GSK3)}) for GSK3, as compared to another kinase and more typically they exhibit a selectivity of at least about 5-fold. Usually, GSK3 inhibitors of the present invention exhibit a selectivity for GSK3, as compared to at least one other kinase, of at least about 10-fold, desirably at least about 100-fold, and more preferably, at least about 1000-fold.

GSK3 inhibitory activity can be readily detected using the assays described herein, as well as assays generally known to those of ordinary skill in the art. Exemplary methods for identifying specific inhibitors of GSK3 include both cell-free and cell-based GSK3 kinase assays. A cell-free GSK3 kinase assay detects inhibitors that act by direct interaction with the polypeptide GSK3, while a cell-based GSK3 kinase assay may identify inhibitors that function either by direct interaction with GSK3 itself, or by interference with GSK3 expression or with post-translational processing required to produce mature active GSK3.

In general, a cell-free GSK3 kinase assay can be readily carried out by: (1) incubating GSK3 with a peptide substrate, radiolabeled ATP (such as, for example, γ³³P- or γ³²P-ATP, both available from Amersham, Arlington Heights, Ill.), magnesium ions, and optionally, one or more candidate inhibitors; (2) incubating the mixture for a period of time to allow incorporation of radiolabeled phosphate into the peptide substrate by GSK3 activity; (3) transferring all or a portion of the enzyme reaction mix to a separate vessel, typically a microtiter well that contains a uniform amount of a capture ligand that is capable of binding to an anchor ligand on the peptide substrate; (4) washing to remove unreacted radiolabeled ATP; then (5) quantifying the amount of ³³P or ³²P remaining in each well. This amount represents the amount of radiolabeled phosphate incorporated into the peptide substrate. Inhibition is observed as a reduction in the incorporation of radiolabeled phosphate into the peptide substrate.

Suitable peptide substrates for use in the cell free assay may be any peptide, polypeptide or synthetic peptide derivative that can be phosphorylated by GSK3 in the presence of an appropriate amount of ATP. Suitable peptide substrates may be based on portions of the sequences of various natural protein substrates of GSK3, and may also contain N-terminal or C-terminal modifications or extensions including spacer sequences and anchor ligands. Thus, the peptide substrate may reside within a larger polypeptide, or may be an isolated peptide designed for phosphorylation by GSK3.

For example, a peptide substrate can be designed based on a subsequence of the DNA binding protein CREB, such as the SGSG-linked CREB peptide sequence within the CREB DNA binding protein described in Wang et. al., Anal. Biochem., 220:397-402 (1994), incorporated herein by reference. In the assay reported by Wang et. al., the C-terminal serine in the SXXXS motif of the CREB peptide is enzymatically prephosphorylated by cAMP-dependent protein kinase (PKA), a step which is required to render the N-terminal serine in the motif phosphorylatable by GSK3. As an alternative, a modified CREB peptide substrate can be employed which has the same SXXXS motif and which also contains an N-terminal anchor ligand, but which is synthesized with its C-terminal serine prephosphorylated (such a substrate is available commercially from Chiron Technologies PTY Ltd., Clayton, Australia). Phosphorylation of the second serine in the SXXXS motif during peptide synthesis eliminates the need to enzymatically phosphorylate that residue with PKA as a separate step, and incorporation of an anchor ligand facilitates capture of the peptide substrate after its reaction with GSK3.

Generally, a peptide substrate used for a kinase activity assay may contain one or more sites that are phosphorylatable by GSK3, and one or more other sites that are phosphorylatable by other kinases, but not by GSK3. Thus, these other sites can be prephosphorylated in order to create a motif that is phosphorylatable by GSK3. The term “prephosphorylated” refers herein to the phosphorylation of a substrate peptide with non-radiolabeled phosphate prior to conducting a kinase assay using that substrate peptide. Such prephosphorylation can conveniently be performed during synthesis of the peptide substrate.

The SGSG-linked CREB peptide can be linked to an anchor ligand, such as biotin, where the serine near the C terminus between P and Y is prephosphorylated. As used herein, the term “anchor ligand” refers to a ligand that can be attached to a peptide substrate to facilitate capture of the peptide substrate on a capture ligand, and which functions to hold the peptide substrate in place during wash steps, yet allows removal of unreacted radiolabeled ATP. An exemplary anchor ligand is biotin. The term “capture ligand” refers herein to a molecule which can bind an anchor ligand with high affinity, and which is attached to a solid structure. Examples of bound capture ligands include, for example, avidin- or streptavidin-coated microtiter wells or agarose beads. Beads bearing capture ligands can be further combined with a scintillant to provide a means for detecting captured radiolabeled substrate peptide, or scintillant can be added to the captured peptide in a later step.

The captured radiolabeled peptide substrate can be quantitated in a scintillation counter using known methods. The signal detected in the scintillation counter will be proportional to GSK3 activity if the enzyme reaction has been run under conditions where only a limited portion (e.g., less than 20%) of the peptide substrate is phosphorylated. If an inhibitor is present during the reaction, GSK3 activity will be reduced, and a smaller quantity of radiolabeled phosphate will thus be incorporated into the peptide substrate. Hence, a lower scintillation signal will be detected. Consequently, GSK3 inhibitory activity will be detected as a reduction in scintillation signal, as compared to that observed in a negative control where no inhibitor is present during the reaction.

A cell-based GSK3 kinase activity assay typically utilizes a cell that can express both GSK3 and a GSK3 substrate, such as, for example, a cell transformed with genes encoding GSK3 and its substrate, including regulatory control sequences for the expression of the genes. In carrying out the cell-based assay, the cell capable of expressing the genes is incubated in the presence of a compound of the present invention. The cell is lysed, and the proportion of the substrate in the phosphorylated form is determined, e.g., by observing its mobility relative to the unphosphorylated form on SDS PAGE or by determining the amount of substrate that is recognized by an antibody specific for the phosphorylated form of the substrate. The amount of phosphorylation of the substrate is an indication of the inhibitory activity of the compound, i.e., inhibition is detected as a decrease in phosphorylation as compared to the assay conducted with no inhibitor present. GSK3 inhibitory activity detected in a cell-based assay may be due, for example, to inhibition of the expression of GSK3 or by inhibition of the kinase activity of GSK3.

Thus, cell-based assays can also be used to specifically assay for activities that are implicated by GSK3 inhibition, such as, for example, inhibition of tau protein phosphorylation, potentiation of insulin signaling, and the like. For example, to assess the capacity of a GSK3 inhibitor to inhibit Alzheimer's-like phosphorylation of microtubule-associated protein tau, cells may be co-transfected with human GSK3β and human tau protein, then incubated with one or more candidate inhibitors. Various mammalian cell lines and expression vectors can be used for this type of assay. For instance, COS cells may be transfected with both a human GSK3β expression plasmid according to the protocol described in Stambolic et. al., 1996, Current Biology 6:1664-68, which is incorporated herein by reference, and an expression plasmid such as pSG5 that contains human tau protein coding sequence under an early SV40 promoter. See also Goedert et. al., EMBO J., 8: 393-399 (1989), which is incorporated herein by reference. Alzheimer's-like phosphorylation of tau can be readily detected with a specific antibody such as, for example, AT8, which is available from Polymedco Inc. (Cortlandt Manor, N.Y.) after lysing the cells.

Likewise, the ability of GSK3 inhibitor compounds to potentiate insulin signaling by activating glycogen synthase can be readily ascertained using a cell-based glycogen synthase activity assay. This assay employs cells that respond to insulin stimulation by increasing glycogen synthase activity, such as the CHO-HIRC cell line, which overexpresses wild-type insulin receptor (˜100,000 binding sites/cell). The CHO-HIRC cell line can be generated as described in Moller et. al., J. Biol. Chem., 265:14979-14985(1990) and Moller et. al., Mol. Endocrinol., 4:1183-1191 (1990), both of which are incorporated herein by reference. The assay can be carried out by incubating serum-starved CHO-HIRC cells in the presence of various concentrations of compounds of the present invention in the medium, followed by cell lysis at the end of the incubation period. Glycogen synthase activity can be detected in the lysate as described in Thomas et. al., Anal. Biochem., 25:486-499 (1968). Glycogen synthase activity is computed for each sample as a percentage of maximal glycogen synthase activity, as described in Thomas et. al., supra, and is plotted as a function of candidate GSK3 inhibitor concentration. The concentration of candidate GSK3 inhibitor that increased glycogen synthase activity to half of its maximal level (i.e., the EC₅₀) can be calculated by fitting a four parameter sigmoidal curve using routine curve fitting methods that are well known to those having ordinary skill in the art.

GSK3 inhibitors can be readily screened for in vivo activity such as, for example, using methods that are well known to those having ordinary skill in the art. For example, candidate compounds having potential therapeutic activity in the treatment of type 2 diabetes can be readily identified by detecting a capacity to improve glucose tolerance in animal models of type 2 diabetes. Specifically, the candidate compound can be dosed using any of several routes prior to administration of a glucose bolus in either diabetic mice (e.g. KK, db/db, ob/ob) or diabetic rats (e.g. Zucker Fa/Fa or GK). Following administration of the candidate compound and glucose, blood samples are removed at preselected time intervals and evaluated for serum glucose and insulin levels. Improved disposal of glucose in the absence of elevated secretion levels of endogenous insulin can be considered as insulin sensitization and can be indicative of compound efficacy.

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, sulfate, 3-phenylpropionate, picrate, pivalate, propionate, succinate, tartrate, thiocyanate, p-toluenesulfonate and undecanoate. Also, the basic nitrogen-containing groups can be quatemized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

Compounds of the present invention can be administered in a variety of ways including enteral, parenteral, inhalation and topical routes of administration. For example, suitable modes of administration include oral, subcutaneous, transdermal, transmucosal, iontophoretic, intracerebral, intravenous, intraarterial, intramuscular, intraperitoneal, intranasal, intrathecal, subdural, rectal, and the like.

In accordance with other embodiments of the present invention, there is provided a composition comprising GSK3-inhibitor compound of the present invention, together with a pharmaceutically acceptable carrier or excipient.

Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference.

Pharmaceutical compositions containing GSK-3 inhibitor compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

The compounds of the present invention may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

In accordance with yet other embodiments, the present invention provides methods for inhibiting GSK3 activity in a human or animal subject, said method comprising administering to a subject an amount of a GSK3 inhibitor compound having the structure (I), (IV), (V) or (VI) (or composition comprising such compound) effective to inhibit GSK3 activity in the subject. Other embodiments provided methods for treating a cell or a GSK3-mediated disorder in a human or animal subject, comprising administering to the cell or to the human or animal subject an amount of a compound or composition of the invention effective to inhibit GSK3 activity in the cell or subject. Preferably, the subject will be a human or non-human animal subject. Inhibition of GSK3 activity includes detectable suppression of GSK3 activity either as compared to a control or as compared to expected GSK3 activity.

Effective amounts of the compounds of the invention generally include any amount sufficient to detectably inhibit GSK3 activity by any of the assays described herein, by other GSK3 kinase activity assays known to those having ordinary skill in the art or by detecting an alleviation of symptoms in a subject afflicted with a GSK3-mediated disorder.

GSK3-mediated disorders that may be treated in accordance with the invention include any biological or medical disorder in which GSK3 activity is implicated or in which the inhibition of GSK3 potentiates signaling through a pathway that is characteristically defective in the disease to be treated. The condition or disorder may either be caused or characterized by abnormal GSK3 activity. Representative GSK3-mediated disorders include, for example, type 2 diabetes, Alzheimer's disease and other neurodegenerative disorders, obesity, atherosclerotic cardiovascular disease, essential hypertension, polycystic ovary syndrome, syndrome X, ischemia, especially cerebral ischemia, traumatic brain injury, bipolar disorder, immunodeficiency, cancer and the like.

Successful treatment of a subject in accordance with the invention may result in the inducement of a reduction or alleviation of symptoms in a subject afflicted with a medical or biological disorder to, for example, halt the further progression of the disorder, or the prevention of the disorder. Thus, for example, treatment of diabetes can result in a reduction in glucose or HbA1c levels in the patient. Likewise, treatment of Alzheimer's disease can result in a reduction in rate of disease progression, detected, for example, by measuring a reduction in the rate of increase of dementia.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present invention, a therapeutically effective dose will generally be from about 0.1 mg/kg/day to about 100 mg/kg/day, preferably from about 1 mg/kg/day to about 20 mg/kg/day, and most preferably from about 2 mg/kg/day to about 10 mg/kg/day of a GSK3 inhibitor compound of the present invention, which may be administered in one or multiple doses.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

While the compounds of the invention can be administered as the sole active pharmaceutical agent, they can also be used in combination with one or more other agents used in the treatment of disorders. Representative agents useful in combination with the compounds of the invention for the treatment of type 2 diabetes include, for example, insulin, troglitazone, rosiglitazone, pioglitazone, glipizide, metformin, acarbose, and the like. Representative agents useful in combination with the compounds of the invention for the treatment of Alzheimer's disease include, for example, donepezil, tacrine and the like. Representative agents useful in combination with the compounds of the invention for the treatment of bipolar disease include, for example, lithium salts, valproate, carbamazepine and the like. A representative agent useful in combination with the compounds of the invention for the treatment of stroke is, for example, tissue plasminogen activator.

When additional active agents are used in combination with the compounds of the present invention, the additional active agents may generally be employed in therapeutic amounts as indicated in the Physicians' Desk Reference (PDR) 53^{rd} Edition (1999), which is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

The foregoing and other aspects of the invention may be better understood in connection with the following representative examples.

### EXAMPLE 1

To determine whether Wnt10b inhibits adipogenesis in vivo, we created transgenic mice that express Wnt10b under control of the fatty acid binding protein-4 (FABP4) promoter. Similar phenotypes are observed in three founder lines. FABP4-Wnt10b founders, (C57BL/6 X SJL)F₂, were backcrossed to C57BL/6 and progeny in N2 to N4 generations were used for experiments. Wnt10b from FABP4 promoter is selectively expressed in white and brown adipose tissues, as well as bone marrow. Male and female FABP4-Wnt10b mice have increased body mass compared to wild type littermates. Metabolic analyses revealed that food intake is similar in wild type and FABP4-Wnt10b mice; however, FABP4-Wnt10b mice consume 7.4% less oxygen. Measurements of tissue weights established that increased body mass of FABP4-Wnt10b is almost exclusively due to greater skin weight, including hair (6.0±0.6 g in transgenic males vs 3.9±0.3 g in wild type males at eight weeks of age). While epidermal and muscle layers in skin appear grossly normal, a dramatic expansion of the dermal layer, coincident with a lack of adipocytes and decreased subcutaneum, is observed in FABP4-Wnt10b mice. Thus, within the dermis, Wnt10b stimulates proliferation of collagen-secreting cells and inhibits adipogenesis.

In addition to decreased adipocyte number in skin, FABP4-Wnt10b mice have less total body fat when fed a low-fat (44% decrease, P<0.05) or a high-fat diet (46% decrease, P<0.01), as assessed by dual energy x-ray absorptiometry. Likewise, epididymal fat pads are smaller in line B FABP4-Wnt10b mice fed a low-fat (40% decrease, P<0.06) or high-fat diet (47% decrease, P<0.001), and similar results are observed with perirenal adipose tissue. Expression of adipocyte markers such as C/EBPα, PPARγ appears to be similar between wild type and FABP4-Wnt10b mice. However, concomitant with reduced adipose tissue, FABP4-Wnt10b mice have lower serum leptin compared with wild type mice (2.0 vs. 3.9 ng/ml, P<0.01). Accumulation of lipid is not observed in liver, muscle, or pancreatic β-cells of FABP4-Wnt10b mice at two or six months of age, despite the block to adipose tissue development. Consistent with the well-established relationship between adipose tissue and whole body insulin resistance (Kahn et. al. 2000), FABP4-Wnt10b mice have improved glucose tolerance and insulin sensitivity at eight weeks of age. Moreover, FABP4-Wnt10b mice resist the glucose intolerance caused by feeding a high-fat diet for 20 weeks. Thus, Wnt10b inhibits development of white adipose tissue and protects against diet-induced obesity and glucose intolerance.

To investigate further the developmental roles of Wnt10b, we created mice with a deletion of the Wnt10b open reading frame. Newborn Wnt10b null mice occur with the expected Mendelian frequency and show no obvious growth or reproductive defects. On a congenic FVB background, Wnt10b −/− and wild type mice have similar amounts of epididymal adipose tissue, underscoring that expansion of adipose tissue occurs as a result of increased food intake and/or decreased total body energy expenditure, rather than unregulated adipogenesis. However, as inhibition of Wnt signaling in C2C12 myoblasts results in spontaneous adipogenesis, and Wnt10b mRNA decreases with age in myoblasts, coincident with increased adipocyte differentiation (Taylor-Jones et. al. 2002), we investigated Wnt10b as a switch between adipogenesis and myogenesis. We utilized a freeze-injury model in which satellite cells are activated, and in wild type mice, rapidly proliferate and differentiate to regenerate myofibers (Pavlath et. al. 1998). In Wnt10b −/− mice, however, activated myoblasts accumulate lipid and express an adipocyte marker, FABP4. Similar results are observed when tibialis muscle is injured with cardiotoxin. Adipogenesis of satellite cells is only observed when Wnt10 −/− mice are fed a high fat diet, suggesting that a stimulus to undergo adipogenesis is also required.

We also examined the role of Wnt10b in development of brown adipose tissue (BAT). BAT is crucial for adaptive thermogenesis in rodents, human infants, and potentially in human adults (Lowell and Spiegelman 2000). In wild type mice, a large BAT depot is observed in the interscapular region, dorsal to the vertebrae, as a lobed tissue stained dark red. Brown adipocytes are enriched for mitochondria, and contain small multilocular triacylglycerol-filled vacuoles. In contrast, interscapular tissue from FABP4-Wnt10b mice contains cells histologically similar to white adipocytes, with large unilocular triacylglycerol-filled vacuoles and displaced nuclei. Enlargement of lipid droplets is observed in other mouse models in which development or function of BAT has been impaired (Enerback et. al., 1997; Thomas et. al. 1997; Moitra et. al. 1998; and Shimomura et. al. 1998). To further characterize interscapular tissue of FABP4-Wnt10b mice, expression of various adipocyte markers were examined. Although FABP4-Wnt10b mice have interscapular tissue that resembles white adipose tissue, adipogenic transcription factors, C/EBPα and PPARγ, and the adipocyte fatty acid binding protein, FABP-4, are not expressed. Moreover, expression of important brown adipocyte genes (Lowell and Spiegelman 2000; Rosen et. al. 2000), such as PGC-1α, PGC-1β, UCP-1 and β3-adrenergic receptor, is also greatly reduced. Finally, FABP4-Wnt10b mice are unable to maintain core body temperature when placed at 4° C., with loss of thermoregulatory control within 72 hours. Taken together, these data indicate that Wnt10b blocks development and function of BAT.

In bone marrow, Wnt signaling may determine whether mesenchymal progenitors differentiate into adipocytes or osteoblasts. Whereas Wnt10b inhibits adipogenesis and adipose tissue development, activation of canonical Wnt signaling stimulates osteoblastogenesis and bone formation (Bain, et. al. 2003; Gong et. al. 2001; Boyden et. al. 2002). However, an endogenous Wnt involved in bone development has not yet been identified. Thus, we investigated the skeletal phenotype of FABP4-Wnt10b and Wnt10b null mice.

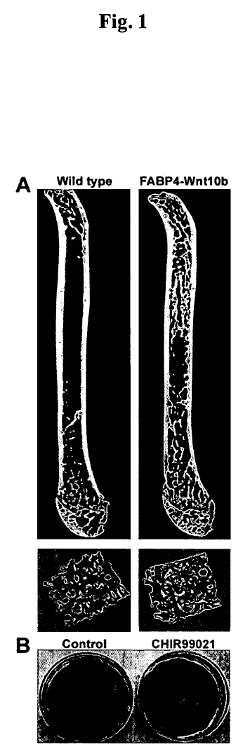

Analysis of FABP4-Wnt10b mice with micro-computed tomography revealed extensive trabecular bone throughout the entire endocortical bone comparment. This bone phenotype is present in both sexes and is observed as early as 10 weeks of age. Trabecular bone volume fraction (BV/TV) in distal femur is increased approximately four-fold (15.8 vs 3.7%, P<0.001) compared to wild type controls, and the distal metaphyseal trabeculae are increased in number (Tb.N.; 4.71 vs 1.43, P<0.001), thickness (Tb.Th.; 0.033 vs 0.024 mm, P<0.05) and are more tightly spaced (Tb.Sp.; 0.19 vs 0.95 mm, P<0.001) (Table 1). Analysis of a 3 cm midcortical segment revealed an increase in bone cross-sectional area, cortical thickness, and bending moments; however, diaphyseal analysis is complicated by the high trabecular content. Mechanical testing by four point bending indicates that femurs from FABP4-Wnt10b mice have increased ultimate load (42.8 vs. 32.0 N, P<0.01) and stiffniess (326.6 vs. 235.4 N/mm, P<0.01) compared to wild type littermates. Effects of Wnt10b are not restricted to femur, as FABP4-Wnt10b mice have increased bone in tibia, humerus, and vertebrae. Increased trabecular bone in FABP4-Wnt10b mice strongly supports the hypothesis that Wnt10b shifts development of mesenchymal precursors from adipogenesis towards osteoblastogenesis. Although increased development of bone could be due, in part, to a reduction in serum leptin (Takeda et. al. 2002), a direct effect of Wnt signaling is likely given that activation of Wnt signaling with a glycogen synthase kinase 3 inhibitor (CHIR99021) increases osteoblastogenesis and mineralization of bipotential ST2 cells (FIG. 1B).

Table 1. Wnt10b increases bone formation and strength in FABP4-Wnt10b mice. Micro-computerized tomography of distal femur from wild type (n=6) and FABP4-Wnt10b (n=6) mice was performed as described (Hankenson et. al. 2000), and analyzed with the Stereology function of GE Medical Systems Microview software. A 1 mm³ region, corresponding to the region highlighted in FIG. 1A, lower panel, was analyzed. Material properties of femurs were evaluated with a Servohydraulic Testing machine (810 Material Test System; Eden Prairie, Minn.) as described (Hankenson et. al. 2000).

| | Wild type | FABP4-Wnt10b | P value |
|---|---|---|---|
| Morphometric | | | |
| properties | | | |
| Trabecular thickness | 0.0244 ± | 0.0329 ± 0.0055 | P < 0.05 |
| (Tb. Th.; mm) | 0.0043 | | |
| Trabecular spacing | 0.95 ± 0.36 | 0.188 ± 0.035 | P < 0.001 |
| (Tb. Sp.; mm) | | | |
| Trabecular number | 1.43 ± 0.59 | 4.71 ± 0.55 | P < 10⁻⁵ |
| (Tb. N.) | | | |
| Material properties | | | |
| Bone mineral den- | 108 ± 63 | 293 ± 85 | P < 0.01 |
| sity (mg/cc) | | | |
| Ultimate load (N) | 32.1 ± 2.9 | 42.8 ± 5.9 | P < 0.01 |
| Stiffness (N/mm) | 235 ± 19 | 327 ± 66 | P < 0.01 |
| Yield Load (N) | 21.0 ± 3.9 | 25.6 ± 7.6 | NS |
| Energy (Nmm) | 11.5 ± 6.2 | 9.8 ± 3.6 | NS |
| Displacement ratio | 2.90 ± 0.5 | 3.33 ± 2.1 | NS |

To determine whether endogenous Wnt10b stimulates osteoblastogenesis, we investigated bone development in Wnt10b −/− mice. Analysis of distal metaphyseal femur revealed that bone volume fraction is decreased by 30% in male Wnt10b −/− mice (Table 2). Bone mineral density and trabecular number are comparably decreased (Table 2). Similar results are observed in female Wnt10b −/− mice. Taken together, results from Wnt10b transgenic and null mice provide compelling evidence that Wnt10b regulates bone development.

Table 2. Wnt10b −/− mice have decreased bone mass and trabecular number. Micro-computed tomography of distal femur from wild type (n=8) and Wnt10b −/− (n=8) mice was performed as described (Hankenson et. al. 2000), and analyzed with the Stereology function of GE Medical Systems Microview software.

| Morpho- | | | | |
|---|---|---|---|---|
| metric pro- | | | | |
| perties | Wild type | Wnt10b−/− | % change | P value |
| Bone miner- | 212 ± 15 | 164 ± 23 | −23 | <0.001 |
| al density | | | | |
| (mg/cc) | | | | |
| Bone volume | 9.23 ± 1.9 | 6.45 ± 1.85 | −30 | <0.01 |
| fraction (BV/ | | | | |
| TV; %) | | | | |
| Bone sur- | 71.6 ± 4 | 74.5 ± 8.2 | +4 | NS |
| face/volume | | | | |
| (BS/BV; | | | | |
| mm⁻¹) | | | | |
| Trabec- | 0.030 ± 0.002 | 0.029 ± 0.003 | −6 | NS |
| ular thick- | | | | |
| ness (Tb. | | | | |
| Th.; mm) | | | | |
| Trabecular | 2.91 ± 0.5 | 2.15 ± 0.51 | −26 | <0.01 |
| number (Tb. | | | | |
| N) | | | | |
| Trabecular | 0.343 ± 0.082 | 0.559 ± 0.272 | +63 | <0.05 |
| spacing (Tb. | | | | |
| Sp.; mm) | | | | |

Expression of Wnt10b from the FABP4 promoter inhibits development of adipose tissues and increases formation and strength of bone. FABP4-Wnt10b mice are resistant to diet-induced obesity and show improved glucose tolerance. Wnt10b deficiency decreases trabecular bone volume, and predisposes activated myoblasts to undergo adipogenesis rather than myogenesis. These results show that for multipotent mesenchymal progenitors, Wnt10b governs the switch between adipogenesis and alternative cell fates, such as osteoblast or myocyte differentiation.

### EXAMPLE 2

Preparation of (VI): 6-[(2-{[4-(2,4-dichlorophenyl)-5-(4-methylimidazol-2-yl)pyrimidin-2-yl]amino}ethyl)amino]pyridine-3-carbonitrile 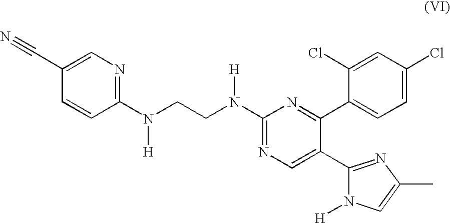
1. Preparation of 1-(2,4-dichlorophenyl)-2-(4-methylimidazol-2-yl)ethan-1-one.

A solution of 2,4-dichlorobenzoyl chloride (7.24 M) in dichloromethane (25 ml) was added dropwise over 20 minutes to a stirred solution of 2,4-dimethylimidazole (0.80 M) in dichloromethane (75 ml) and N,N-diisopropylethylamine (Hünig's base) (34 ml). The reaction mixture was cooled during the addition using a water bath. The reaction mixture was then heated to reflux for 5 hours. The reaction can turn a darker color. The product was stripped of solvent under reduced pressure, and the resulting solid was dried in vacuo for one hour.

To the dry solid (described above) was added a solution (2:1 v/v, 120 ml) of gla. acetic acid and aq. con. HCl. The mixture was then stirred at reflux for ca. 90 min. The acetic acid was removed via rotary evaporator. Upon cooling, distilled water (200 ml) and toluene (100 ml) were added to the solid residue, which was vigorously stirred for 30 min. The solids were filtered, rinsed with 50 ml distilled water, and discarded. The filtrate was transferred to a separatory funnel. After the organic layer was discarded, the aqueous layer was washed with toluene (2×100 ml). The aqueous layer was transferred to a large beaker (2 L) and diluted with isopropyl ether (50 ml). The stirred mixture was basified (pH 7-8) by careful addition of sodium bicarbonate which leads to the formation of a sticky white solid. Dichloromethane (200 ml) was added and stirring continued for 10 min. The organic layer was separated and the aqueous layer was again extracted with dichloromethane (100 ml). The organic layers were combined and washed with sat. aq. NaHCO3 (100 ml), distilled water (100 ml), brine (100 ml), dried with Na2SO4, filtered, concentrated, and dried in vacuo giving 1-(2,4-dichlorophenyl)-2-(4-methylimidazol-2-yl)ethan-1-one in 46% yield.

2. Preparation of (2Z)-1-(2,4-dichlorophenyl)-3-(dimethylamino)-2-(4-methylimidazol-2-yl)prop-2-en-1-one.

A mixture of 1-(2,4-dichlorophenyl)-2-(4-methylimidazol-2-yl)ethan-1-one (0.33 M) and N,N-dimethylformamide-dimethyl acetal (DMFDMA) (25 ml) was stirred for 2.5 h at 70-75° C. The DMFDMA was then removed under reduced pressure and dried under high vacuum for several hours giving a light orange solid in quantitative yield. The enaminone product (2Z)-1-(2,4-dichlorophenyl)-3-(dimethylamino)-2-(4-methylimidazol-2-yl)prop-2-en-1-one was typically used without further purification.

3. Preparation of 6-[(2-aminoethyl)amino]pyridine-3-carbonitrile.

A mixture of 2-chloro-5-cyanopyridine (0.60 M) in acetonitrile (120 ml) and ethylene diamine (85 ml) were stirred overnight (ca. 16 h) at 75-80° C. under argon. The ethylene diamine was removed under reduced pressure and then dried in vacuo for 2-3 h. The residual solution was basified with 1M sodium hydroxide solution (˜100 ml). The aqueous solution was saturated with sodium chloride and extracted with a solution of 95% ethyl acetate and 5% methanol (3×150 ml) and with a solution of 95% acetonitrile and 5% methanol (3×150 ml). The organic extracts were combined and extracted with a saturated sodium chloride solution (2×70 ml). The organic layer was dried with sodium sulfate, filtered, and concentrated under reduced pressure. The crude white to tan solid was triturated with ether (2×50 ml) and dried overnight in vacuo resulting in 78% yield of 6-[(2-aminoethyl)amino]pyridine-3-carbonitrile.

4. Preparation of amino{2-[(5-cyano(2-pyridyl))amino]ethyl}carboxamidine, hydrochloride.

A mixture of 6-[92-aminoethyl)amino]pyridine-3-carbonitrile (0.47 M), 1H-pyrazole-1-carboxamidine hydrochloride (0.47 M), and acetonitrile (120 ml) were stirred ca. 24 h at 75-80° C. Upon cooling, a precipitate was collected by filtration. The white solid was washed thoroughly with acetonitrile (2×100 ml), ethyl ether (3×100 ml), and dried overnight in vacuo giving amino{2-[(5-cyano(2-pyridyl))amino]ethyl}carboxamidine as the HCl salt in 82% yield.

5. Preparation of 6-[(2-{[4-(2,4-dichlorophenyl)-5-(4-methylimidazol-2-yl)pyrimidin-2-yl]amino}ethyl)amino]pyridine-3-carbonitrile.

A solution of sodium ethoxide (0.58 M) dissolved in abs. ethanol (15 ml) was added to a stirred mixture of (2Z)-1-(2,4-dichlorophenyl)-3-(dimethylamino)-2-(4-methylimidazol-2-yl)prop-2-en-1-one (0.41 M), amino{2-[(5-cyano(2-pyridyl))amino]ethyl}carboxamidine, hydrochloride (0.43 M), and abs. ethanol (20 ml). The reaction was then heated to 75-80° C. for 2.5 hours. On cooling the reaction was diluted with ethyl acetate (400 ml) washed with sat. aq. NaHCO₃ (100 ml), distilled water (2×100 ml), brine (100 ml), dried with Na₂SO₄, filtered, and concentrated. The crude product (˜50% purity) was purified by flash chromatography over silica gel. The column was run starting with 1:1 ethyl acetate to hexane, then ethyl acetate which was used until all of the fast moving impurities had been removed. The product was eluted with 1.5% methanol in ethyl acetate. The column is monitored by TLC using 5% methanol in ethyl acetate as the solvent system. The product has UV activity in the long wave length region and “glows” blue on the unstained TLC plate. The proper fractions were condensed. The off-white solid was dried overnight in vacuo giving 6-[(2-{[4-(2,4-dichlorophenyl)-5-(4-methylimidazol-2-yl)pyrimidin-2-yl]amino}ethyl)amino]pyridine-3-carbonitrile in 28% yield.

HPLC: 20.7 min (>99% purity)

MS: M+H=465.3 (C₂₂H₁₈C₁₂N₈+H=465)

1. A method of treating or preventing bone loss in a human or animal subject, comprising administering to the human or animal subject a compound of formula (I): 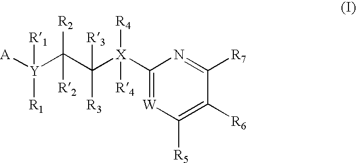
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R′₁, R′₂, R′₃ and R′₄ are independently selected from the group consisting of hydrogen, and optionally substituted loweralkyl;
R₅ and R₇ are independently selected from the group consisting of hydrogen, halo, and optionally substituted loweralkyl, cycloalkyl, alkoxy, amino, aminoalkoxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido; or
pharmaceutically acceptable salts thereof, stereoisomers thereof, tautomers thereof, hydrates thereof, or solvates thereof.

2. The method of claim 1 wherein said compound is:

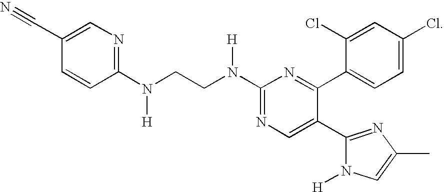

3. The method of claim 1 wherein the bone loss is related to osteopenia, osteoporosis, drug therapy, postmenopausal bone loss, age, disuse, diet, rheumatism, rheumatoid arthritis, Paget's disease, periodontal disease, cancer, cancer treatment, or bone fracture.

4. The method of claim 3 wherein said bone fracture is a hip or spinal fracture.

5. The method of claim 3 wherein said drug therapy is administration of a steroid.

6. The method of claim 3 wherein said cancer is multiple myeloma, breast, prostate, or lung cancer.

7. The method of claim 1 wherein said compound is further administered in combination with at least one additional agent for the treatment or prevention of bone loss.

8. The method of claim 7 wherein said additional agent is estrogen or calcium.

9. The method of claim 7 wherein said additional agent is an anti-resorption agent.

10. The method of claim 9 wherein said anti-resorption agent is selected from the group consisting of raloxifene, calcitonin, alendronate, clodronate, etidronate, pamidronate, ibandronate, zoledronic acid, risedronate, and tiludronate.

11. The method of claim 7 wherein said additional agent is an osteogenic promoting agent.

12. The method of claim 11 wherein said osteogenic promoting agent is a parathyroid hormone.

13. The method of claim 1 wherein the treatment promotes bone formation.

14. A composition comprising a compound of formula (I) and at least one additional agent for the treatment or prevention of bone loss, wherein 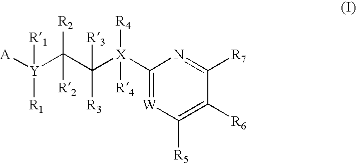
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R′₁, R′₂, R′₃ and R′₄ are independently selected from the group consisting of hydrogen, and optionally substituted loweralkyl;
R₅ and R₇ are independently selected from the group consisting of hydrogen, halo, and optionally substituted loweralkyl, cycloalkyl, alkoxy, amino, aminoalkoxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido; or
pharmaceutically acceptable salts thereof, stereoisomers thereof, tautomers thereof, hydrates thereof, or solvates thereof.

15. The composition of claim 14 wherein said additional agent is estrogen or calcium.

16. The composition of claim 14 wherein said additional agent is an anti-resorption agent.

17. The composition of claim 16 wherein said anti-resorption agent is selected from the group consisting of raloxifene, calcitonin, alendronate, clodronate, etidronate, pamidronate, ibandronate, zoledronic acid, risedronate, and tiludronate.

18. The composition of claim 14 wherein said additional agent is an osteogenic promoting agent.

19. The composition of claim 18 wherein said osteogenic promoting agent is a parathyroid hormone.

20. The composition of claim 14 wherein said compound is:

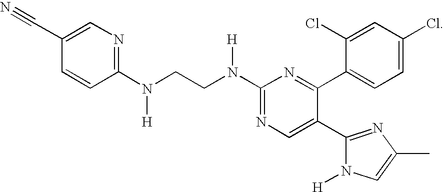

21. The use of a compound in the manufacture of a medicament for the treatment or prevention of a bone loss, said compound having the formula (I): 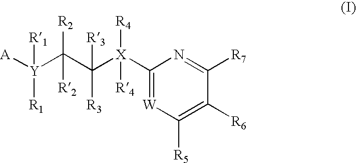
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R′₁, R′₂, R′₃ and R′₄ are independently selected from the group consisting of hydrogen, and optionally substituted loweralkyl;
R₅ and R₇ are independently selected from the group consisting of hydrogen, halo, and optionally substituted loweralkyl, cycloalkyl, alkoxy, amino, aminoalkoxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido; or
pharmaceutically acceptable salts thereof, stereoisomers thereof, tautomers thereof, hydrates thereof, or solvates thereof.

22. The use of the compound of claim 21, wherein said compound is: [chemical structure]
